(19) United States
(12) Patent Application Publication
(10) Pub. No.: US 2025/0041800 A1
(43) Pub. Date: Feb. 6, 2025

(54) RECOMBINANT MICROORGANISM HAVING ENHANCED NITROGEN OXIDE REDUCTION ABILITY INCLUDING GENETIC MODIFICATION INCREASING EXPRESSION OF ELECTRON-TRANSFER PROTEIN GENE, AND USE THEREOF

(71) Applicant: Samsung Electronics Co., Ltd., Suwon-si (KR)

(72) Inventors: Yu Kyung Jung, Suwon-si (KR); Jae-Young Kim, Suwon-si (KR); Jinha Kim, Suwon-si (KR); Seung Hoon Song, Suwon-si (KR); Woo Yong Shim, Suwon-si (KR)

(21) Appl. No.: 18/410,386

(22) Filed: Jan. 11, 2024

(30) Foreign Application Priority Data
Aug. 2, 2023 (KR) 10-2023-0101119
Jan. 8, 2024 (KR) 10-2024-0003127

(51) Int. Cl. B01D 53/84; B01D 53/56; C12N 1/20; C12N 9/06; C12N 9/12; C12N 15/70

(52) U.S. Cl. CPC B01D 53/84; B01D 53/56; C12N 1/20; C12N 9/0044; C12N 9/1205; C12N 15/70; C12Y 107/02004; C12Y 207/01; B01D 2257/404; B01D 2258/06; C12N 2800/101

(57) ABSTRACT

A recombinant microorganism having enhanced ability for reducing nitrogen oxide, a composition comprising the recombinant microorganism for use in decreasing a concentration of nitrogen oxide in a sample, and a method of decreasing a concentration of nitrogen oxide in a sample using the recombinant microorganism or the composition.

### CROSS-REFERENCE TO RELATED APPLICATION

This application is based on and claims priority under 35 U.S.C. § 119, and all the benefits accruing therefrom, to Korean Patent Application No. 10-2023-0101119 filed on Aug. 2, 2023, and Korean Patent Application No. 10-2024-0003127 filed on Jan. 8, 2024 in the Korean Intellectual Property Office, the contents of which are incorporated by reference herein in its entirety.

### BACKGROUND

### 1. Field

The disclosure relates to a recombinant microorganism having enhanced ability for reducing nitrogen oxide, a composition including the recombinant microorganism for use in decreasing a concentration of nitrogen oxide in a sample, and a method of decreasing a concentration of nitrogen oxide in a sample.

### 2. Description of the Related Art

Nitrogen oxides (NOₓ) are one of the air pollutants mainly emitted during the combustion process of fuels. Nitrogen oxides include nitric oxide (NO), nitrous oxide (N₂O), N₂O₃, NO₃⁻ (nitrate), NO₂⁻ (nitrite), N₂O₄, and N₂O₅, and the like. Among these nitrogen oxides, nitric oxide and nitrous oxide are mainly responsible for air pollution. Nitrous oxide absorbs and stores heat in the atmosphere together with carbon dioxide (CO₂), methane (CH₄), and Freon gas (e.g. fluorochlorocarbons (CFCs)), thereby causing the greenhouse effect. Nitrous oxide is one of the six greenhouse gases subject to regulation by the Kyoto Protocol. Nitrous oxide has a global warming potential (GWP) of 310, which has a higher warming effect per unit mass than carbon dioxide and methane. In addition, nitrogen oxide is also a leading cause of smog and acid rain, and nitrogen oxide undergoes chemical reactions in the air to form secondary fine dust and increases ground-level ozone concentration, which adversely affects respiratory health.

Most nitrogen oxide removal processes are chemical reduction methods, such as a selective catalytic reduction (SCR) method and a selective non-catalytic reduction (SNCR) method, and techniques using scrubbing, adsorption, and the like are being applied. Chemical methods have drawbacks such as energy and catalyst costs required in the entire process, treatment of secondary waste generated therefrom, and the like. Further, in the case of an SCR or SNCR, another greenhouse gas, nitrous oxide, may be generated as a result of incomplete reduction during reducing nitric oxide and nitrous oxide. Unlike chemical technologies having such problems, biological processes are environmentally friendly processes with advantages such as a relatively simple principle, no use of extreme conditions (including, e.g. high temperature and high pressure), and low generation of secondary waste or wastewater. In such biological processes, microorganisms serving as biological catalysts are used instead of chemical catalysts to oxidize or reduce NOx or to fix NOx as a part of cells.

Denitrifying microorganisms reduce nitrogen oxide to nitrogen through a dissimilatory reductive process. Through several past studies, many denitrifying microorganisms, such as Pseudomonas putida, Pseudomonas denitrificans, Pseudomonas stutzeri, Paracoccus denitrificans, and Klebsiella pneumonia, have been reported.

However, even according to the related art described above, alternative methods of biological denitrification arestill required.

### SUMMARY

In one aspect is provided a recombinant microorganism of the genus Escherichia, including a genetic modification that increases expression of an electron-transfer protein gene or/and a genetic modification that increases expression of a nosZ gene encoding nitrous oxide reductase NosZ, a nosR gene encoding NosR, a nosD gene encoding NosD, a nosF gene encoding NosF, a nosY gene encoding NosY, and an apbE gene encoding ApbE, which are derived from microorganisms of the genus Pseudomonas or Paracoccus.

In another aspect is provided a composition for use in decreasing a concentration of nitrogen oxide in a sample, the composition including a recombinant microorganism including a genetic modification that increases expression of an electron-transfer protein gene and a genetic modification that increases expression of a nosZ gene encoding nitrous oxide reductase NosZ, a nosR gene encoding NosR, a nosD gene encoding NosD, a nosF gene encoding NosF, a nosY gene encoding NosY, and an apbE gene encoding ApbE, which are derived from microorganisms of the genus Pseudomonas or Paracoccus.

In yet another aspect is provided a method of decreasing a concentration of nitrogen oxide in a sample, the method including: decreasing a concentration of nitrogen oxide in a sample by contacting a recombinant microorganism of the genus Escherichia with a sample including nitrogen oxide, the recombinant microorganism including a genetic modification that increases expression of an electron-transfer protein gene or/and a genetic modification that increases expression of a nosZ gene encoding nitrous oxide reductase NosZ, a nosR gene encoding NosR, a nosD gene encoding NosD, a nosF gene encoding NosF, a nosY gene encoding NosY, and an apbE gene encoding ApbE, which are derived from microorganisms of the genus Pseudomonas or Paracoccus.

Additional aspects will be set forth in part in the description which follows and, in part, will be apparent from the description, or may be learned by practice of the presented embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings.

FIG. 1A is a diagram showing a concentration of intermediate nitrous oxide over time, as a result of converting Fe(II)EDTA-NO to nitrogen by using a recombinant E. coli DeNOx strain in which a nitrous oxide reduction pathway is introduced into the chromosome.

FIG. 1B is a diagram showing a concentration of a final nitrogen product over time, as a result of converting Fe(II)EDTA-NO to nitrogen by using a recombinant E. coli DeNOx strain in which a nitrous oxide reduction pathway is introduced into the chromosome.

FIG. 2 is a diagram showing ¹⁵N₂ produced when recombinant DeNOx E. coli strains including a genetic modification that introduces one of 10 electron-transfer protein genes and increases expression thereof is reacted in the presence of Fe(II)EDTA-¹⁵NO. DeNOx/pTrc99a is a control strain not having an introduced electron-transfer protein gene.

FIG. 3A is a diagram showing a concentration of intermediate nitrous oxide over time when recombinant DeNOx E. coli strains including a genetic modification that introduces expression of one of four electron-transfer protein genes is reacted in the presence of Fe(II)EDTA-¹⁵NO. DeNOx/pTrc99a is a control strain not having an introduced electron-transfer protein gene.

FIG. 3B is a diagram showing a concentration of a final nitrogen product over time when recombinant DeNOx E. coli strains including a genetic modification that introduces expression of one of four electron-transfer protein genes is reacted in the presence of Fe(II)EDTA-¹⁵NO. DeNOx/pTrc99a is a control strain not having an introduced electron-transfer protein gene.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

The terminology used herein is for the purpose of describing particular embodiments only and is not intended to be limiting. As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise. “Or” means “and/or.” “At least one” is not to be construed as limiting “a” or “an.” As used herein, the term “and/or” includes any and all combinations of one or more of the associated listed items. As used herein, the term “or” and “and/or” includes any and all combinations of one or more of the associated listed items. Expressions such as “at least one of,” when preceding a list of elements, modify the entire list of elements and do not modify the individual elements of the list.

It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±20%, 10% or ±5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The term “derived from” as used herein includes “originating/originate from” and “obtained from”.

The term “increase in expression” as used herein refers to a detectable increase in expression of a given gene or protein. The term “increased expression” refers to an increased expression level of a given gene or protein in a modified cell (e.g., a genetically engineered cell) compared to that in a comparative cell of the same type without a given genetic modification (e.g., a native cell or a “wild-type” cell). For example, the expression level of the gene or protein in the modified cell may be increased by about 5% or more, about 10% or more, about 15% or more, about 20% or more, about 30% or more, about 50% or more, about 60% or more, about 70% or more, or about 100% or more, than the expression level of the gene or protein in an unengineered cell of the same type, for example, a wild-type cell or a parent cell. Cells with increased expression of a protein or an enzyme may be identified using any method known in the art.

The term “copy number increase” as used herein refers to an increase in copy number caused by introduction or amplification of a given gene, and may also include a case in which a gene not present in an unengineered cell is obtained by genetic engineering. The introduction of a gene may be achieved through a vehicle such as a vector. The introduction may include transient introduction in which a gene is not integrated into the genome, or insertion of a gene into the genome. The introduction may be achieved by, for example, introducing a vector into which a polynucleotide encoding a desired polypeptide is inserted into a cell, and then replicating the vector in the cell or integrating the polynucleotide into the genome. The term “increase in copy number” may refer to, when several polypeptides form a complex and exhibit one activity, an increase in the copy number of one or more genes encoding one or more polypeptides constituting the complex. A plurality of genes may be introduced. For example, the number of introduced genes may be 2 or more, 5 or more, 10 or more, 20 or more, 50 or more, 100 or more, or 1,000 or more.

The introduction of a gene may be performed by any known method, such as transformation, transfection, electroporation, or gene editing. Here, a gene may be introduced through a vehicle, or may be introduced as it is. The term “vehicle” as used in this context refers to a nucleic acid molecule capable of delivering another nucleic acid linked to the vehicle. In terms of a nucleic acid sequence that mediates the introduction of a particular gene, the vehicle as used in this context is understood to be used interchangeably with a vector, a nucleic acid construct, and a cassette. A vector may be, for example, a plasmid-derived vector or a virus-derived vector. A plasmid may include a circular double-stranded DNA loop to which additional DNA may be ligated. A vector may be, for example, a plasmid expression vector or a viral expression vector including, for example, a replication-defective retrovirus, an adenovirus, or an adeno-associated virus.

The term “decrease in expression” as used herein refers to a detectable decrease in expression of a given gene or protein. The term “decreased expression” as used herein refers to an expression level of a given gene or protein in a modified (e.g., genetically engineered) cell that is lower than that in a comparative cell of the same type without a given genetic modification (e.g., the original or “wild-type” cell). For example, the expression level of the given gene or protein in the modified cell may be decreased by about 5% or greater, about 10% or greater, about 15% or greater, about 20% or greater, about 30% or greater, about 50% or greater, about 60% or greater, about 70% or greater, or about 100% or greater, as compared with that in an unmodified cell, e.g., a wild-type cell. A cell having decreased expression of a protein or enzyme may be identified by using a method known to those of ordinary skill in the art.

A decrease in expression of a gene or protein may be caused by, e.g., deletion or disruption of a gene encoding a protein. A deletion or disruption of a gene may be a genetic modification that results in no expression or reduced expression of the gene, or no activity or reduced activity of the protein encoded by the gene, which may also result from inactivation or attenuation of expression of the gene.

A genetic modification may include, e.g., a part or all of a gene having a modification, or a part or all of a regulatory factor of a gene, such as a promoter thereof or a terminator region thereof, having a modification. A genetic modification may include, for example, mutation, substitution, deletion, or insertion of one or more nucleotides of, from or into a gene, or any combination thereof. Deletion or disruption of a gene may be achieved through gene manipulation, such as homologous recombination, mutagenesis induction, or molecular evolution. A genetic modification may include substitution of an endogenous promoter with a stronger or weaker promoter. When a cell contains a plurality of identical genes or contains two or more paralogs, one or more genes may be deleted or disrupted.

Genetic engineering may be performed by a molecular biological method known in the art.

The term “parent cell” as used herein refers to an original cell, for example, a non-genetically engineered cell of the same type relative to a genetically engineered cell or a genetically engineered microorganism. In terms of a particular genetic modification harbored by a genetically engineered cell, the “parent cell” is a cell that does not have the particular genetic modification, but may have the same or similar characteristics as the genetically engineered cell otherwise. Thus, the parent cell is a cell that may be used as a starting material to produce a genetically engineered cell having an increased or decreased expression level of a given gene or protein (e.g., a protein having at least about 75%, 80%, 85%, 90%, 95% or greater amino acid sequence identity to a Fur protein).

The term “gene” as used herein refers to a nucleic acid sequence encoding a particular protein, or may be non-protein coding, and may or may not include a regulatory sequence with a 5′ non-coding sequence and/or a 3′ non-coding sequence.

A “polypeptide” is a polymer chain comprised of amino acid residue monomers which are joined together through amide bonds (peptide bonds). In general, a polypeptide may include at least 10, 20, 50, 100, 200, 500, or more amino acid residue monomers.

The term “sequence identity” of a nucleic acid/polynucleotide or a polypeptide as used herein refers to the degree of sameness or identicalness of nucleic acid or amino acid residues between sequences after aligning both sequences to be as identical as possible in a specific comparison region. The sequence identity is a value measured by optimally aligning and comparing two sequences in a specific comparison region, and a portion of the sequences in the comparison region may be added or deleted compared to a reference sequence. A sequence identity percentage may be, for example, calculated by steps of: comparing two optimally aligned sequences throughout the comparison region; determining the number of positions in both sequences where identical amino acids or nucleotides appear to obtain the number of matched positions; dividing the number of the matched positions by the total number of positions within the comparison range (i.e., a range size); and multiplying the result by 100 to obtain a sequence identity percentage. A sequence identity percentage may be determined by using a sequence comparison program known in the art, examples of which include BLASTN (NCBI), BLASTP (NCBI), CLC Main Workbench (CLC bio), MegAlign™ (DNASTAR Inc), and the like.

The term “genetic modification” as used herein refers to an artificial alteration in a sequence, a composition or a structure of a genetic material.

One aspect provides a recombinant microorganism of the genus Escherichia including a genetic modification that increases expression of an electron-transfer protein gene or/and a nosZ gene encoding nitrous oxide reductase NosZ.

In the recombinant microorganism, the electron-transfer protein may serve to transfer electrons to an acceptor protein. The electron-transfer protein may include c-type cytochrome, cupredoxin, or a combination thereof.

The c-type cytochrome may include heme c covalently attached to the peptide backbone through one or two thioether bonds. The c-type cytochrome may have a binding motif specific to Cys-XX-Cys-His (CXXCH). The X may be any amino acid. The c-type cytochrome may belong to class 1. The class 1 is a small, soluble c-type cytochrome protein with a molecular weight of 8 to 12 kDa, and may have a single heme group. The class 1 can be subclassified into Classes IA to IE. The c-type cytochrome may include cytochromes c, c1, c2, c5, c555, c550-553, c556, c6, and cbb3. The c-type cytochrome may be Pseudomonas stutzeri (Ps)-derived cytochrome c5 (Ps cyt C5), Pseudomonas stutzeri (Ps)-derived cytochrome c (Ps cyt C), Pseudomonas stutzeri (Ps)-derived cytochrome c551 (Ps cyt c551), Pseudomonas stutzeri(Ps)-derived cytochrome c550 (Ps cyt c550), Pseudomonas aeruginosa (Pa)-derived cytochrome c550 (Pa cyt c550), or Marinobacter nauticus (Mn)-derived cytochrome c552 (Mn cyt c552), or a combination thereof.

The Ps cyt c5, the Ps cyt c, the Ps cyt c551, the Ps cyt c550, the Pa cyt c550, and the Mn cyt c552 may be polypeptides having a sequence identity of 75% or greater, e.g. 80% or greater, 85% or greater, 90% or greater, 95% or greater, or 98% or greater, to the amino acid sequences of SEQ ID NOs: 1, 3, 5, 7, 9, and 11, respectively.

The Ps cyt c5, the Ps cyt c, the Ps cyt c551, the Ps cyt c550, the Pa cyt c550, and the Mn cyt c552 may be encoded by the nucleotide sequences of SEQ ID NOs: 2, 4, 6, 8, 10, and 12, respectively.

The cupredoxin may be a small, soluble protein including type-I copper in its active site. The cupredoxin is also called blue copper protein. The cupredoxin may have a molecular weight of about 10 kDa to about 14 kDa. The cupredoxin may belong to the subfamilies of plastocyanin, azurin, pseudoazurin, amicyanin, rusticyanin, cucumber basic proteins, and stellacyanins, depending on high-resolution X-ray and NMR structures.

In an embodiment, the cupredoxin may belong to the subfamilies of azurin, the pseudoazurin, and the amicyanin. Although cupredoxins belonging to different subfamilies often show a low sequence identity to each other, they may have an eight-stranded Greek key β-barrel or β-sandwich fold and a highly conserved active site architecture.

The azurin may be a small, periplasmic, bacterial blue copper protein found in Pseudomonas, Bordetella, or Alcaligenes bacteria. The azurin may regulate single-electron transfer between cytochrome-bound enzymes by causing oxidation-reduction between Cu(I) and Cu(II). Each monomer of azurin tetramer may have a molecular weight of about 14 kDa and include a single copper atom. In addition, each monomer may be strongly blue and have a fluorescence emission band centered at 308 nm.

The pseudoazurin may have a “plastocyanin-like” fold structure with two α-helices in the C-terminal region. The azurin and the pseudoazurin show different overall electrostatic properties from each other, but may have similar electrostatic properties at the copper site, especially at the copper site in a reduced state. The azurin and the pseudoazurin may exhibit different binding specificities for electron donors or electron acceptors, for example, Cu—NIR.

The overall fold of the amicyanin is very similar to that of plastocyanin, but there may be a large extension at the N-terminus of amicyanin.

The pseudoazurin may include Achromobacter cycloclastes (Ac)-derived pseudoazurin (Ac paz), Sinorhizobium meliloti (Sm)-derived pseudoazurin (Sm paz), or Paracoccus versutus (Pv)-derived pseudoazurin (Pv paz), or a combination thereof. The amicyanin may be Pv-derived amicyanin (Pv ami).

The Ac paz, the Sm paz, the Pv paz, and the Pv ami may be polypeptides having a sequence identity of 75% or greater, e.g., 80% or greater, 85% or greater, 90% or greater, 95% or greater, or 98% or greater, to the amino acid sequences of SEQ ID NOs: 13, 15, 17, and 19, respectively.

The Ac paz, the Sm paz, the Pv paz, and the Pv ami may be encoded by the nucleotide sequences of SEQ ID NOs: 14, 16, 18, and 20, respectively.

The genetic modification that increases expression of a gene encoding an electron transport protein may be an increase in the copy number of the gene encoding the electron transport protein. The copy number of the gene may be increased by introducing the gene into a chromosome. This introduction may be achieved by homologous recombination, gene editing, or directed mutagenesis. A starting strain or parental strain used to introduce a gene may not contain the gene or may not express the gene.

The recombinant microorganism may further include a genetic modification that decreases the expression of one or more of fur gene, nsrR gene, and lac gene.

Fur, NsrR, and Lac may be polypeptides having a sequence identity of 75% or greater, for example, 80% or greater, 85% or greater, 90% or greater, 95% or greater, or 98% or greater, to the amino acid sequences of SEQ ID NOs: 21, 23, and 25, respectively. The Fur, the NsrR, and the Lac may be encoded by the nucleotide sequences of SEQ ID NOs: 22, 24, and 26, respectively.

The genetic modification may include destruction or removal of one or more genes among the fur gene, the nsrR gene, and the lac gene.

The Fur protein may function as a ferric uptake regulator, a transcriptional dual regulator, or a repressor of iron acquisition genes when iron is sufficient. By decreased expression of the fur gene, the transport of FeEDTA-NO into cells may be increased.

The NsrR is a nitric oxide-sensitive regulator, serving as a global transcriptional regulator in response to reactive nitrogen species (RNS) in E. coli. By decreased expression of the nsrR gene, cells may be protected from reactive nitrogen species such as nitric oxide.

The Lac is a transcriptional repressor of a lac-based promoter, and may suppress the expression of nos genes designed to be regulated by a tac promoter, which is one type of a lac-based promoter. To induce expression, an inducer such as isopropyl β-D-1-thiogalactopyranoside (IPTG) is required. By decreased expression of the lac gene, nos genes, which undergo transcriptional repression by the Lac, may be expressed without a supply of IPTG.

The recombinant microorganism may further include a genetic modification that adjusts or increases, or both adjusts and increases, expression of ccmABCDEFGH operon. The biosynthesis of c-type cytochromes in gram-negative bacteria may rely on 8 cytochrome c maturation (ccm) proteins. These proteins are all located in the periplasm, and may be anchored or integral to the cytoplasmic membrane. The maturation of c-type cytochromes is a post-translational process that requires reduction conditions, and occurs in the periplasm, which is an oxidizing environment. In E. coli, the expression of the 8 ccm proteins may be regulated by the ccmABCDEFGH operon. The term “increasing the expression of the ccmABCDEFGH operon” refers to synthesizing c-type cytochromes in an active form not only under anaerobic conditions but also under aerobic conditions, by adjusting or increasing, or both adjusting and increasing, the expression of the 8 genes included in the operon. The adjusting or increasing, or both adjusting and increasing, the expression of the 8 ccm proteins ABCDEFGH may enable the production of exogenous c-type cytochromes under both aerobic and anaerobic conditions.

The genetic modification may include substitution of an endogenous promoter of E. coli with a stronger promoter to constitutively express the 8 ccm protein genes. The genetic modification may include substitution of an endogenous promoter of E. coli with a stronger promoter, such as a Trc promoter, a Tac promoter, or a T7 promoter. In addition, the genetic modification may include increasing the number of copies of the 8 ccm protein genes. The endogenous promoter of the ccmABCDEFGH operon may have the nucleotide sequence of SEQ ID NO: 27.

The recombinant microorganism may have a genetic modification that increases the expression of the nosZ gene encoding nitrous oxide reductase NosZ. The genetic modification may include introducing the nosZ gene into a starting strain or parent strain that does not include or express the nosZ gene. The NosZ may be derived from a microorganism. The NosZ may be derived from a microorganism of the genus Pseudomonas or the genus Paracoccus. The microorganism of the genus Pseudomonas may be P. stutzeri or P. aeruginosa. The microorganism of the genus Paracoccus may be P. versutus.

The recombinant microorganism may have an increased ability to reduce nitrogen oxides, compared to its ability to reduce nitrogen oxides before the genetic modification that increases the expression of the nosZ gene is introduced. By introducing a nosZ gene to a starting strain or parent strain that does not include or express the nosZ gene and increasing expression of the nosZ gene therein, the recombinant microorganism may have a newly conferred or increased ability to reduce nitrogen oxides. Such ability may be conferred by the genetic modification. The ability to reduce nitrogen oxides may include reducing nitric oxide (NO) to nitrous oxide (N₂O) and reducing nitrous oxide (N₂O) to nitrogen (N₂), or a combination thereof.

The recombinant microorganism may further include one or more genetic modification that increases expression of a nosR gene encoding NosR, a nosL gene encoding NosL, a nosD gene encoding NosD, a nosF gene encoding NosF, a nosY gene encoding NosY, and an apbE gene encoding ApbE, or a combination thereof wherein the genes are derived from microorganisms of the genus Pseudomonas or Paracoccus. These genes may be introduced into a starting strain or parent strain that does not include or express these genes, and the expression of these genes may be induced or increased in the resulting recombinant microorganism.

In one aspect, the recombinant microorganism may belong to the genus Escherichia and have a genetic modification that increases the expression of an electron-transfer protein gene or/and a genetic modification that increases the expression of genes derived from the genus Pseudomonas or the genus Paracoccus, such as a nosZ gene encoding nitrous oxide reductase NosZ, a nosR gene encoding NosR, a nosD gene encoding NosD, a nosF gene encoding NosF, a nosY gene encoding NosY, and an apbE gene encoding ApbE, or a combination thereof.

In some aspects, the recombinant microorganism may belong to the genus Escherichia and have a genetic modification that increases the expression of one or more electron-transfer protein genes or/and a genetic modification that increases the expression of one or more genes derived from the genus Pseudomonas or the genus Paracoccus, such as a nosZ gene encoding nitrous oxide reductase NosZ, a nosR gene encoding NosR, a nosL gene encoding NosL, a nosD gene encoding NosD, a nosF gene encoding NosF, a nosY gene encoding NosY, or an apbE gene encoding ApbE, or a combination thereof.

In some aspects, the electron-transfer protein may be c-type cytochrome or/and cupredoxin. The electron-transfer protein may be Pseudomonas stutzeri (Ps)-derived cytochrome c5 (Ps cyt c5), Pseudomonas stutzeri (Ps)-derived cytochrome c (Ps cyt c), Pseudomonas stutzeri (Ps)-derived cytochrome c551 (Ps cyt c551), Pseudomonas stutzeri (Ps)-derived cytochrome c550 (Ps cyt c550), Pseudomonas aeruginosa (Pa)-derived cytochrome c550 (Pa cyt c550), Marinobacter nauticus (Mn)-derived cytochrome c552 (Mn cyt c552), Achromobacter cycloclastes (Ac)-derived pseudoazurin (Ac paz), Sinorhizobium meliloti (Sm)-derived pseudoazurin (Pv paz), Paracoccus versutus (Pv)-derived pseudoazurin (Pv paz), or Paracoccus versutus (Pv)-derived amicyanin (Pv ami), or a combination thereof. In addition, the microorganism of the genus Pseudomonas or the genus Paracoccus may be P. stutzeri, P. aeruginosa, or P. versutus. The recombinant microorganism of the genus Escherichia may be E. coli.

The nitrous oxide reductase may be NosZ, a product of the nosZ gene, which catalyzes a reaction of converting nitrous oxide (N₂O) to nitrogen (N₂). NosZ may be a homodimeric metalloprotein of 130 kDa that contains two copper centers, CuA and CuZ, in each monomer. The NosR may be encoded by the nosR gene, and may be a polytopic membrane protein serving as an electron donor for the nitrous oxide reduction.

The NosL is a membrane-anchored copper chaperone that binds Cu¹⁺ for delivery to apo-NosZ. The NosL may be encoded by the nosL gene.

NosD may be encoded by the nosD gene, and may be essential for the formation of [4Cu:2S] CuZ site. NosD may supply sulfur (S) to NosZ. NosF and NosY may be encoded by the nosF gene and the nosY gene, respectively, and may form a complex, such as a tetramer, to serve as an ABC transporter. ApbE may be encoded by the apbE gene, and may be a flavinyltransferase that transfers flavin to NosR.

The recombinant microorganism may include a genetic modification that increases the expression of a gene encoding an enzyme that catalyzes the reaction of converting nitric oxide (NO) to nitrous oxide. Such an enzyme may be nitric oxide reductase (NOR). An example of this enzyme may be NorV encoded by a norV gene. The NorV may be di-ion center-containing flavorubredoxin-type NOR. The NorV may catalyze a reaction of converting nitric oxide (NO) or Fe(II)(L)-NO to nitrous oxide (N₂O). The genetic modification may include increasing the number of copies of the norV gene.

The genetic modification may include increasing the copy number of one or more of the nosZ gene, the nosR gene, the nosD gene, the nosL gene, the nosF gene, the nosY gene, and the apbE gene.

The genetic modification may include introduction of the genes, for example, introduction of the genes through a vehicle such as a vector. The genes may be present intrachromosomally or extrachromosomally. The genes may be introduced into a chromosome through homologous recombination. Multiple copies of the introduced genes may be present. For example, the copy number of the introduced genes may independently be 2 or more, 5 or more, 10 or more, 20 or more, 50 or more, 100 or more, or 1,000 or more.

The NosZ, NosR, NosL, NosD, NosF, NosY, and ApbE may be polypeptides having a sequence identity of 75% or more, for example, 80% or more, 85% or more, 90% or more, 95% or more, or 98% or more, to the amino acid sequences of SEQ ID NOs: 28, 30, 32, 34, 36, 38, and 40, respectively. The nosZ gene, nosR gene, nosL gene, nosD gene, nosF gene, nosY gene, and apbE gene may have the nucleotide sequences of SEQ ID NOs: 29, 31, 33, 35, 37, 39, and 41, respectively.

In the recombinant microorganism, 6 genes including the nosZ gene, nosR gene, nosD gene, nosF gene, nosY gene, and apbE gene, or 7 genes including the nosZ gene, nosR gene, nosL gene, nosD gene, nosF gene, nosY gene, and apbE gene, may be introduced to the recombinant microorganism through a vector. The vector may exist outside a chromosome, e.g. extrachromosomal. Alternatively, the 6 or 7 genes may be introduced into a chromosome by genetically engineered methods including homologous recombination.

In the recombinant microorganism, the nosZ gene, the nosR gene, and the nosL gene may be operably linked to one strong promoter, the nosD gene and the nosY gene may be operably linked to one strong promoter, and the apbE gene and the nosF gene may be operably linked to one strong promoter, or the linked construct may be introduced into a chromosome. The strong promoter may be, for example, a tac promoter. The recombinant microorganism may be one in which operons are introduced into a chromosome by homologous recombination at lacI, fur, and nsrR gene regions, wherein the operons may include: a Ptac-nosZRL operon in which the nosZ gene, nosR gene, and nosL gene are operably linked to one tac promoter; a Ptac-nosDY operon in which the nosD gene and the nosY gene are operably linked to one tac promoter; and a Ptac-apbE-nosF operon in which the apbE gene and the nosF gene are operably linked to one tac promoter. The lacI, fur, and nsrR genes may be inactivated or deleted.

In the recombinant microorganism, the nosZ gene, the nosR gene, and the nosL gene may be included in one operon, the nosD gene and the nosY gene may be included in one operon, and the apbE gene and the nosF gene may be included in one operon.

The recombinant microorganism may be capable of decreasing a level of nitrogen oxides (NOx) in a sample by reducing nitrogen oxides. The recombinant microorganism may have an increased ability to reduce the level of nitrogen oxides in the sample. The nitrogen oxides may include nitrous oxide (N₂O), nitric oxide (NO), N₂O₃, NO₃⁻ (nitrate), NO₂⁻ (nitrite), N₂O₄, N₂O₅, or a mixture thereof. The nitrogen oxides may be in free or complexed form. The complexed form may be in the form of Fe(II)(L)-nitrogen oxide. The Fe(II)(L)-nitrogen oxide (NOx) may be a chelating complex of chelating agent L, Fe²⁺, and NOx. The Fe(II)(L)-nitrogen oxide may be in the form of Fe(II)(L)-NO. The Fe(II)(L)-NO may be a chelating complex of chelating agent L, Fe²⁺, and nitric oxide (NO). L may be, for example, ethylenediamine, diethylenetriamine, triethylenetetramine, hexamethylenetetramine, N-(2-hydroxyethyl)ethylenediamine-triacetic acid (HEDTA), ethylenediamine-tetraacetic acid (EDTA), iminodiacetic acid, nitrilo-triacetic acid (NTA), or diethylenetriaminepentaacetic acid (DTPA). Therefore, the Fe(II)(L)-NO may be in a modified form such that nitrogen oxide, such as nitrous oxide (N₂O), nitric oxide (NO), N₂O₃, NO₃⁻ (nitrate), NO₂⁻ (nitrite), N₂O₄, or N₂O₅, is soluble in an aqueous solution. The formation of Fe(II)(L)-NOx, e.g., Fe(II)(L)-NO, may be achieved by contacting an aqueous solution containing Fe(II)(L) with nitrogen oxide such as nitric oxide (NO). The contacting may include mixing an aqueous medium with liquid nitrogen oxide or contacting an aqueous medium with gaseous nitrogen oxide. However, the recombinant microorganism is not limited to this specific mechanism in decreasing the concentration of nitrogen oxides, such as nitric oxide or nitrous oxide, in a sample.

The recombinant microorganism may decrease the concentration of nitrogen oxide, such as nitric oxide, in a sample. The decreasing may include: converting Fe(II)(L)-NO to nitrous oxide (N₂O) by a denitrification enzyme such as nitric oxide reductase, or/and converting nitrous oxide (N₂O) to nitrogen (N₂) by a denitrification enzyme such as nitrous oxide reductase. The sample may be in a liquid state or a gaseous state. The sample may be industrial wastewater or waste gas. The sample may be any material that includes a nitrogen oxide such as nitric oxide. The nitrogen oxide may include nitrous oxide (N₂O), nitric oxide (NO), N₂O₃, NO₃⁻ (nitrate), NO₂⁻ (nitrite), N₂O₄, or N₂O₅, or any combination or all thereof. The nitric oxide may be nitric oxide (NO) or Fe(II)(L)-NO.

In another aspect is provided a composition for use in decreasing a concentration of nitrogen oxide in a sample, the composition including a recombinant microorganism of the genus Escherichia including a genetic modification that increases expression of an electron-transfer protein gene or/and a genetic modification that increases expression of a nosZ gene encoding nitrous oxide reductase NosZ.

Regarding the composition, the recombinant microorganism, the sample, and the nitrogen oxide may be the same as those described above.

Regarding the composition, decreasing the concentration of nitrogen oxide, such as nitric oxide or nitrous oxide, in a sample may include complete removal. The sample may be gas or liquid. The composition may further include a material that increases the solubility of nitrogen oxide, such as nitric oxide or nitrous oxide, in a medium or culture. The nitric oxide may be in the form of nitric oxide (NO) or Fe(II)(L)-NO.

The composition may be used to decrease the concentration of nitrogen oxide, such as nitric oxide or nitrous oxide, in a sample by contacting the composition with the sample. The contacting may be performed in a liquid phase. The contacting may be achieved by, for example, contacting the sample with a culture of a recombinant microorganism cultured in a medium. The culture may be achieved under conditions for proliferating the microorganism. The contacting may be performed in a closed container. The contacting may be performed under anaerobic conditions. The contacting may include culturing or incubating the recombinant microorganism in the presence of a sample including nitrogen oxide, such as nitric oxide or nitrous oxide. The contacting may include culturing the recombinant microorganism under conditions for proliferating the recombinant microorganism in a closed container. The medium may be a chemically defined medium. The term “chemically defined medium” as used herein refers to a medium supplemented with a known chemical composition. Such a chemically defined medium may not contain a composite component of unknown chemical composition such as serum or hydrolysate. A liquid medium may include an LB medium, an M9 medium, a phosphate buffer, a Tris buffer, and the like. Such a medium may contain Mg²⁺ ions in a concentration range of about 0.1 mM to about 7.5 mM, about 0.5 mM to about 7.5 mM, about 0.5 mM to about 5.0 mM, about 0.5 mM to about 2.5 mM, about 0.5 mM to about 1.5 mM, or about 1.0 mM to about 2.5 mM.

In another aspect is provided a method of decreasing a concentration of nitrogen oxide in a sample, the method including decreasing a concentration of nitrogen oxide in a sample by contacting a sample having nitrogen oxide with a recombinant microorganism of the genus Escherichia having a genetic modification that increases expression of an electron-transfer protein gene or/and a genetic modification that increases expression of a nosZ gene encoding nitrous oxide reductase NosZ.

Regarding the method, the recombinant microorganism, the nitrogen oxide, or the sample including nitrogen oxide may be the same as those described above.

In the method, the contacting may be performed in a liquid phase. The contacting may be achieved by, for example, contacting the sample with a culture of a recombinant microorganism cultured in a medium. The culture may be achieved under conditions for proliferating the microorganism. The contacting may be performed in a closed container. The contacting may be performed under anaerobic conditions. The medium may be a chemically defined medium. Such a chemically defined medium may not contain a composite component such as serum or hydrolysate. A liquid medium may include an LB medium, an M9 medium, a phosphate buffer, a Tris buffer, and the like. Such a medium may contain Mg²⁺ ions in a concentration range of about 0.1 mM to about 7.5 mM, about 0.5 mM to about 7.5 mM, about 0.5 mM to about 5.0 mM, about 0.5 mM to about 2.5 mM, about 0.5 mM to about 1.5 mM, or about 1.0 mM to about 2.5 mM.

The contacting may be performed when the growth phase of the recombinant microorganism is at an exponential phase or a stationary phase. The culturing may be performed under anaerobic conditions. The contacting may be performed under conditions in which the recombinant microorganism is viable in a closed container. The conditions in which the recombinant microorganism is viable may refer to conditions that allow the recombinant microorganism to remain in a proliferative state.

In the method, the sample may be in a liquid state or a gaseous state. The sample may be industrial wastewater or waste gas. The contacting of sample may include active contacting as well as passive contacting with the culture of the recombinant microorganism. The sample may be, for example, provided by sparging into a medium or culture solution of the microorganism. For example, a gaseous sample may be blown or bubbled through a medium or culture solution of the recombinant microorganism, such as blowing or bubbling from the bottom of the medium or culture solution to the top. Alternatively, the sparging may be injecting or adding a liquid sample into a medium or culture solution of the recombinant microorganism.

In the method, the contacting may be performed in a batch or continuous manner. The contacting may include contacting the sample with fresh recombinant microorganism one or more times. Such contacting with fresh recombinant microorganism may be performed at least twice, for example, twice, 3 times, 5 times, or 10 times. The contacting may be continued or repeated for a period of time until the concentration of nitrogen oxide, such as nitric oxide or nitrous oxide, in the sample is decreased to a desired level.

In another aspect is provided a method of preparing a recombinant microorganism with increased ability to remove nitrogen oxide in a sample, the method including introducing a genetic modification that increases expression of an electron-transfer protein gene or/and introducing a genetic modification that increases expression of a nosZ gene encoding nitrous oxide reductase NosZ to a microorganism. The method may be a method of preparing a microorganism, the method including introducing gene(s) to a microorganism. The introducing of gene(s) may be introducing a vehicle including the gene(s) to the microorganism or introducing the gene(s) into a chromosome by homologous recombination. The method may also include introducing a genetic modification which decreases the expression of the fur gene, the nsrR gene, or the lac gene, or a combination thereof, into a chromosome of the microorganism. The genetic modification may include inactivation or destruction of one or more of the gene(s). Also, the method may include introducing a genetic modification which increases the expression of the ccm ABCDEGFH operon into a chromosome of the microorganism. The genetic modification may include substitution of an endogenous promoter with a stronger promoter. Terms that are the same and not separately defined, including the term “nitrogen oxide”, mean the same as described above.

The recombinant microorganism according to an aspect may be used to remove nitrogen oxide, such as nitric oxide or nitrous oxide, from a sample.

The composition according to another aspect may be used to decrease the concentration of nitrogen oxide, such as nitric oxide or nitrous oxide, in a sample.

The method of decreasing the concentration of nitrogen oxide, such as nitric oxide or nitrous oxide, in a sample according to another aspect may efficiently decrease the concentration of nitrogen oxide, such as nitric oxide or nitrous oxide, in a sample.

Hereinafter, the present disclosure will be described in detail with reference to Examples below. However, these Examples are provided for illustrative purposes only, and the scope of the present disclosure is not limited thereto.

### EXAMPLES

### Example 1: Construction of a Recombinant Denitrifying Microorganism with Enhanced Nitrogen Oxide Reduction Ability and Determination of Nitrogen Oxide Reduction Ability of the Recombinant Denitrification Microorganism

In this Example, nosZ, nosR, nosL, nosD, nosY, nosF, and apbE genes derived from Pseudomonas stutzeri (Ps) were introduced into fur, nsrR, and lac gene regions in E. coli to prepare recombinant E. coli imparted with NOx reduction ability and having enhanced NOx reduction ability.

1. Construction of an E. coli Strain in which nosZ, nosR, nosL, nosD, nosF, nosY, and ApbE Genes Derived from P. stutzeri were Introduced into the Chromosome Along with Deletion of Fur, nsrR, and Lac Genes of E. coli.

In E. coli W3110 strain, Ps nos genes were inserted into the chromosome of E. coli while the fur, nsrR, and lac genes were deleted according to a one-step inactivation method (KA Datsenko and BL Wanner, Proc Natl Acad Sci USA, 2000, June 6; 97(12):6640-5).

A pTacHR vector was prepared by linking a DNA fragment and a vector DNA fragment according to the In-Fusion® method (Clontech Laboratories, Inc.), wherein the DNA fragment was obtained through PCR using DNA of SEQ ID NO: 42 including Ptac synthesized as a template and oligonucleotides of SEQ ID NOs: 43 and 44 as primers, and the vector DNA fragment was obtained through PCR using a pMloxC vector (Lee, K. H. et al., Molecular Systems Biology, 3,149 (2007)) as a template and oligonucleotides of SEQ ID NOs: 45 and 46 as primers.

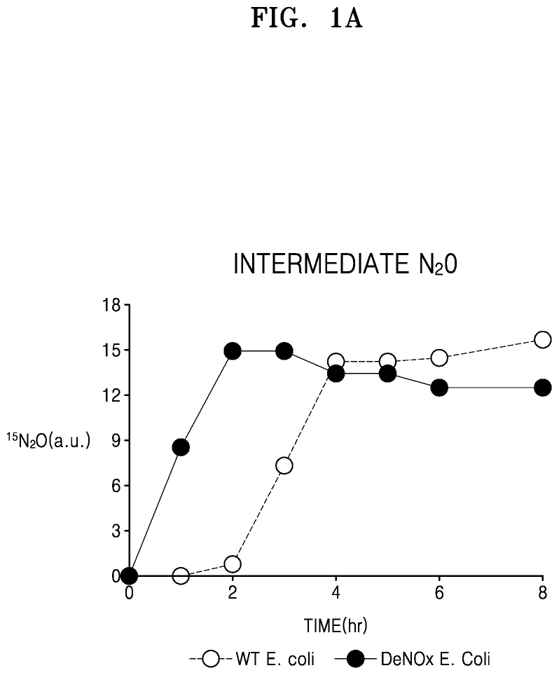

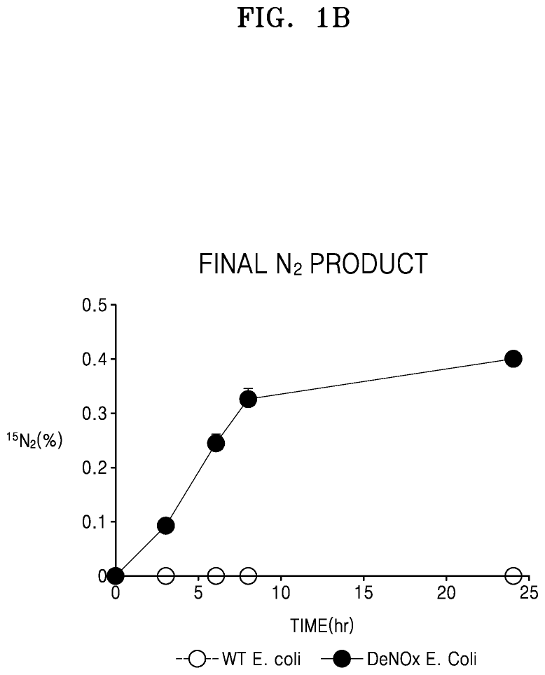

A pTacHR-nosDY vector was prepared by linking a DNA fragment and a vector DNA fragment according to the In-Fusion® method (Clontech Laboratories, Inc.), wherein the DNA fragment was obtained through PCR using the pACYCDuet-nosDY-tat-apbE-nosF vector (FIG. 1 of US 2022/0177896A1) as a template and oligonucleotides of SEQ ID NOs: 47 and 48 as primers, and the vector DNA fragment was obtained through PCR using a pTacHR vector as a template and oligonucleotides of SEQ ID NOs: 49 and 50 as primers. Also, a pTacHR-apbE-nosF vector was prepared by linking a DNA fragment and a vector DNA fragment according to the In-Fusion® method (Clontech Laboratories, Inc.), wherein the DNA fragment was obtained through PCR using the pACYCDuet-nosDY-tat-apbE-nosF vector (FIG. 1 of US 2022/0177896A1) as a template and oligonucleotides of SEQ ID NOs: 51 and 52 as primers, and the vector DNA fragment was obtained through PCR using a pTacHR vector as a template and oligonucleotides of SEQ ID NOs: 49 and 50 as primers.

A pTacHR-nosZRL vector was prepared by linking a DNA fragment and a vector DNA fragment according to the In-Fusion® method (Clontech Laboratories, Inc.), wherein the DNA fragment was obtained through PCR using the pET28a-nosZRL vector (FIG. 1 of US 2022/0177896A1) as a template and oligonucleotides of SEQ ID NOs: 53 and 54 as primers, and the vector DNA fragment was obtained through PCR using a pTacHR vector as a template and oligonucleotides of SEQ ID NOs: 49 and 50 as primers.

Next, PCR was performed by using the pTacHR-nosDY vector as a template and the oligonucleotides of SEQ ID NOs: 55 and 56 as primers. Through this, a DNA fragment designed to have Ptac-nosDY inserted by homologous recombination along with deletion of the fur gene was obtained.

The DNA fragment thus obtained was electroporated to a competent cell of the W3110 strain in which A-red recombinase was expressed, to prepare a mutant strain in which a fur gene was deleted and Ptac-nosDY was inserted. To confirm a mutation in the fur gene region, colony PCR was performed by using primers of SEQ ID NOs: 57 and 58.

In addition, for insertion of Ptac-apbE-nosF along with nsrR deletion, a pTacHR-apbE-nosF vector was used as a template and primers of SEQ ID NOs: SEQ ID NOs: 59 and 60 were used sequentially in the same manner as described above. To determine the nsrR region mutation, primers of SEQ ID NOs: 61 and 62 were used.

In addition, for insertion of Ptac-nosZRL along with lac deletion, a pTacHR-apbE-nosF vector was used as a template and primers of SEQ ID NOs: 63 and 64 were used sequentially in the same manner as described above. To determine the lac region mutation, primers of SEQ ID NOs: 65 and 66 were used.

As a result, strains having the genotypes of W3110Δfur::Ptac-nosDY ΔnsrR::Ptac-apbE-nosF ΔlacI::Ptac-nosZRL were obtained.

Specifically, from P. stutzeri, which is a natural denitrification strain, nosZ gene encoding nitrous oxide reductase (NosZ), which is a key enzyme, and auxiliary genes which are essential for the NosZ to have activity, such as nosR, nosL, nosD, nosF, nosY, and apbE genes, were extracted from a nos operon or gene cluster and the genome, were codon-optimized for E. coli, and then were introduced into E. coli. Accordingly, a recombinant E. coli having the ability to convert nitrogen oxide to nitrogen was obtained.

The function of each product of the introduced genes was considered as follows. NosZ is a product of the nosZ gene, and is an enzyme that catalyzes the conversion of nitrous oxide to nitrogen. That is, NosZ is a nitrous oxide reductase. NosZ may be a homodimeric metalloprotein of 130 kDa that contains two copper centers, CuA and CuZ, in each monomer. NosR may be encoded by the nosR gene and may be a polytopic membrane protein serving as an electron donor for nitrous oxide reduction. NosL is a membrane-anchored copper chaperone that binds Cu¹⁺ for delivery to apo-NosZ. NosL may be encoded by the nosL gene. NosD may be encoded by the nosD gene, and may be essential for the formation of [4Cu:2S] CuZ site. NosD may supply sulfur (S) to NosZ. NosF and NosY may be encoded by the nosF gene and the nosY gene, respectively, and may form a complex, such as a tetramer, to serve as an ABC transporter. ApbE may be encoded by the apbE gene, and may be a flavinyltransferase that transfers flavin to NosR. The NosZ, NosR, NosL, NosD, NosF, NosY, and ApbE used in this example may be polypeptides having amino acid sequences of SEQ ID NOs: 28, 30, 32, 34, 36, 38, and 40, respectively. The nosZ gene, nosR gene, nosL gene, nosD gene, nosF gene, nosY gene, and apbE gene may have the nucleotide sequences of SEQ ID NOs: 29, 31, 33, 35, 37, 39, and 41, respectively. The origin and characteristics of the above-described proteins and the nucleotides encoding the same are set forth in the Sequence Listing. Among the genes inserted into the chromosome of E. coli in this Example, the nucleotide sequences of the native genes that were optimized in consideration of the codon frequency used in E. coli were used, and information thereof is described in the Sequence Listing.

3. Construction of an E. coli Strain Continuously Expressing ccmABCDEFGH Operon

In addition, according to the one-step inactivation method (KA Datsenko and BL Wanner, Proc Natl Acad Sci U.S.A., 2000, June 6; 97(12): 6640-5), the natural promoters of the ccm operon were substituted with trc promoters in the aforementioned W3110Δfur::Ptac-nosDY ΔnsrR::Ptac-apbE-nosF ΔlacI::Ptac-nosZRL strain.

Overlapping PCR using primers of SEQ ID NOs: 67 and 71 was performed on a DNA fragment, which was obtained through PCR using a pMloxC vector (Lee, K. H. et. al., Molecular Systems Biology, 3,149 (2007)) as a template and oligonucleotides of SEQ ID NOs: 67 and 68 as primers, and a DNA fragment, which was obtained through PCR using DNA of SEQ ID NO: 69 including Ptrc as a template and oligonucleotides of SEQ ID NOs: 70 and 71 as primers, to obtain a DNA fragment in which the two DNA fragments were linked. The DNA fragment thus obtained was electroporated into competent cells of the constructed strain (W3110Δfur::Ptac-nosDY ΔnsrR::Ptac-apbE-nosF ΔlacI::Ptac-nosZRL) expressing A-red recombinase to prepare mutant strains in which a promoter of the ccm operon was substituted with a trc promoter. To confirm the mutation of the ccm region, colony PCR using primers of SEQ ID NOs: 72 and 73 was performed.

Accordingly, the ccmABCDEFGH operon was continuously expressed.

As a result, W3110Δfur::Ptac-nosDY ΔnsrR::Ptac-apbE-nosF ΔlacI::Ptac-nosZRL Pccm::Ptrc strain was obtained. Hereinafter, such strain is also referred to as DeNOx strain.

### 4. Confirmation of Nitrogen (N₂) Producibility

The activity of producing nitrogen by reducing nitrogen oxide was confirmed for the prepared DeNOx strain.

### (4.1) Culture for NosZ Maturation

The recombinant E. coli was cultured overnight in 2×YT medium (ThermoFisher Scientific, Waltham, MA] supplemented with 0.25 mM CuCl₂ in an Erlenmeyer flask while stirring at 30° C. at 230 rpm. Next, the cells were harvested and used for subsequent nitrogen production reactions.

(4.2) Nitrogen Production Reaction from Fe(II)EDTA-¹⁵NO

Cells of recombinant E. coli DeNOx strains were added to a M9 medium (pH 7.0) supplemented with 5 g/L glucose and 5 mM Fe(II)EDTA-¹⁵NO until OD₆₀₀ reached 1, thereby obtaining a reaction mixture.

30 mL of the reaction mixture was added to a 60 mL serum bottle and allowed for a reaction while stirring at 30° C. at 140 rpm. The serum bottle was kept in an anaerobic chamber to be maintained under anaerobic conditions. A control was prepared in the same manner, except that wild-type E. coli was used.

Next, the amounts of ¹⁵N₂ and ¹⁵N₂O produced were analyzed by GC-MS by sampling the gas in the headspace of the reaction serum bottle.

The results are shown in FIGS. 1A and 1B. FIG. 1A is a diagram showing the concentration of an intermediate nitrous oxide over time, as a result of converting Fe(II)EDTA-NO to nitrogen (N₂) by using the recombinant E. coli DeNOx strain in which the nitrous oxide reduction pathway was introduced into the chromosome.

FIG. 1B is a diagram showing the concentration of a final nitrogen product over time, as a result of converting Fe(II)EDTA-NO to nitrogen by using the recombinant E. coli DeNOx strain in which the nitrous oxide reduction pathway was introduced into the chromosome.

As shown in FIGS. 1A and 1B, compared to the wild-type E. coli (W3110 strain), use of the DeNOx strain rapidly increased the concentration of intermediate nitrous oxide from Fe(II)EDTA-NO, and the production of nitrogen as the final reduction product was confirmed. In the wild-type E. coli (W3110 strain), nitrogen was not measurable. More specifically, the Fe(II)EDTA-NO was reduced to N₂O in E. coli cells by an endogenous E. coli nitric oxide reductase (NOR), NorV, and in the case of the DeNOx strains, the nitrous oxide was reduced to nitrogen by P. stutzeri-derived nitrous oxide reductase, NosZ.

### Example 2: Construction of a Recombinant Denitrifying Microorganism Having a Genetic Modification that Increases Expression of an Electron Transfer Protein Gene and Determination of Nitrogen Oxide Reduction Ability of the Recombinant Denitrification Microorganism

In this Example, candidate electron-transfer protein genes were introduced into the recombinant microorganism of Example 1 having enhanced nitrogen oxide reduction ability, and the nitrogen oxide reduction ability of obtained strains was confirmed.

1. Construction of a Recombinant Denitrification E. coli Strain to which a Gene for an Electron Transfer Protein was Introduced

The electron transfer proteins were selected with reference to previous literature related to in vitro studies on electron donation of nitrite reductases catalyzing the conversion of NO₂⁻ (nitrite) to NO (nitric oxide), and computer simulations. The electron transfer proteins are shown in Table 1 below.

TABLE 1

| | Denitrifying | Electron transfer | | |
|---|---|---|---|---|
| No. | microorganism | protein | Gene | Mark |
| 1 | Pseudomonas | Cytochrome c5 | PSJM300_ | Ps cyt c5 |
| | stutzeri | | 02355 | |
| 2 | | Cytochrome c | PSJM300_ | Ps cyt c |
| | | | 03220 | |
| 3 | | Cytochrome c551 | PSJM300_ | Ps cyt c551 |
| | | | 03225 | |
| 4 | | Cytochrome c550 | PSJM300_ | Ps cyt c550 |
| | | | 09495 | |
| 5 | Pseudomonas | Cytochrome c550 | exaB | Pa cyt c550 |
| | aeruginosa | | | |
| 6 | Marinobacter | Cytochrome c552 | Cyt c552 | Mn cyt c552 |
| | naticus | | | |
| 7 | Achromobacter | Pseudoazurin | bcp | Ac paz |
| | cycloclasts | | | |
| 8 | Sinorhizobium | Pseudoazurin | azu1 | Sm paz |
| | meliloti | | | |
| 9 | Paracoccus | Pseudoazurin | ATH84_ | Pv paz |
| | versutus | | 103055 | |
| 10 | | Amicyanine | ami | Pv ami |

Sequences including codons optimized for the above 10 genes in E. coli were synthesized. Each gene was amplified by PCR using each of the 10 synthesized genes as a template, and oligonucleotides of SEQ ID NOs: 74 and 75; 76 and 77; 78 and 79; 80 and 81; 82 and 83; 84 and 85; 86 and 87; 88 and 89; 90 and 91; and 92 and 93 were used as primer sets for the respective 10 genes, respectively. Each of the 10 amplified genes was separately linked to a vector fragment according to the In-Fusion® method (Clontech Laboratories, Inc.), wherein the vector fragment was obtained through PCR using the pTrc99a vector (Amersham Pharmacia Biotech) as a template and oligonucleotides of SEQ ID NOs: 94 and 95 as a primer set, so that 10 different pTrc99a-based expression vectors, each containing one of the 10 electron-transfer protein genes operably linked to Ptrc, were obtained. Here, the genes were each operably linked to induce expression thereof by IPTG.

Each of the 10 pTrc99a-based expression vectors was introduced into the DeNOx strain according to the electric shock method (Sambrook, J &Russell, DW, New York: Cold Spring Harbor Laboratory Press, 2001). A list of the resulting recombinant DeNOx E. coli strains to which one of the 10 foreign electron-transfer protein genes was introduced is shown in Table 2 below.

TABLE 2

| No. | Recombinant DeNOx E. coli | Mark | Remark |
|---|---|---|---|
| | DeNOx/pTrc99a | DeNOx/ | [Control] Strain with |
| | | pTrc99a | empty vector |
| 1 | DeNOx/pTrc99a-Ps cyt c5 | Ps cyt c5 | [Experimental group] |
| 2 | DeNOx/pTrc99a-Ps cyt c | Ps cyt c | Recombinant DeNOx |
| 3 | DeNOx/pTrc99a-Ps cyt c551 | Ps cyt c551 | strains in which a foreign |
| 4 | DeNOx/pTrc99a-Ps cyt c550 | Ps cyt c550 | electron-transfer protein |
| 5 | DeNOx/pTrc99a-Pa cyt c550 | Pa cyt c550 | gene was introduced and |
| 6 | DeNOx/pTrc99a-Mn cyt | Mn cyt c552 | overexpressed |
| | c552 | | |
| 7 | DeNOx/pTrc99a-Ac paz | Ac paz | |
| 8 | DeNOx/pTrc99a-Sm paz | Sm paz | |
| 9 | DeNOx/pTrc99a-Pv paz | Pv paz | |
| 10 | DeNOx/pTrc99a-Pv ami | Pv ami | |

2. Confirmation of ¹⁵N₂ Producibility from Fe(II)EDTA-¹⁵NO

Cells of each recombinant DeNOx E. coli strain containing a foreign electron-transfer protein gene were stirred at 230 rpm at 30° C. in a 2×YT medium (Sigma Aldrich, Y2377) supplemented with 100 μg/mL of ampicillin and 0.25 mM CuSO₄. Meanwhile, 0.2 mM IPTG was added thereto to induce expression of the electron-transfer protein gene and the Nos genes. The culture thus obtained was used for the NOx reduction reaction.

The culture was added to a M9 medium (Sigma Aldrich, M6030) (pH 7.0) supplemented with 5 g/L glucose and 5 mM Fe(II)EDTA-¹⁵NO until OD₆₀₀ reached 1, thereby obtaining a reaction mixture.

30 mL of the reaction mixture was added to a 60 mL serum bottle and allowed for a reaction while stirring at 30° C. at 140 rpm for 24 hours. The serum bottle was kept in an anaerobic chamber to be maintained under anaerobic conditions. A control group was prepared in the same manner, except that E. coli including an empty vector was used.

Next, the amounts of ¹⁵N₂ and ¹⁵N₂O produced were analyzed by GC-MS by sampling the gas in the headspace of the reaction serum bottle.

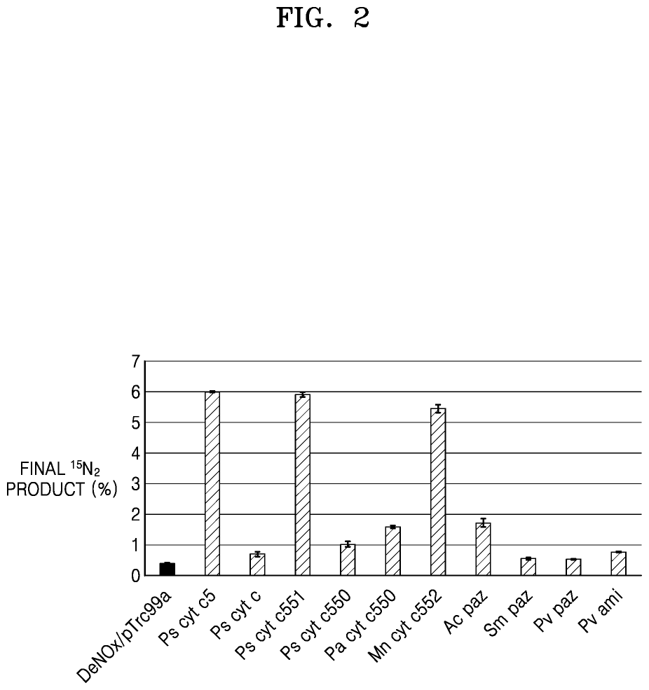

FIG. 2 is a diagram showing ¹⁵N₂ produced when a recombinant DeNOx E. coli strain including a genetic modification that introduces one of the 10 electron-transfer protein genes described above and increases expression thereof was reacted in the presence of Fe(II)EDTA-¹⁵NO. DeNOx/pTrc99a is a control strain with an empty vector.

As shown in FIG. 2, nitrogen (¹⁵N₂) production was increased by at least 1.36 times due to a genetic modification that increases the expression of an electron-transfer protein gene, with Sm paz and Pv paz inducing the smallest amount of nitrogen production. In particular, nitrogen production was increased by about 14.4 times to about 15.8 times in the case of Ps cyt c5, Ps cyt c551, and Mn cyt c552, by about 4.2 times to about 4.5 times in the case of Ac paz and Pa cyt c550, by about 2.0 times to about 2.7 times in the case of Ps cyt c550 and Pv ami, and by about 1.36 times to about 1.80 times in the case of Pv paz, Sm paz, and Ps cyt c. These results show that introduction of a foreign electron-transfer protein gene can remarkably increase the production of nitrogen via reduction of nitrogen oxide.

In addition, the four strains producing the greatest amount of nitrogen in FIG. 2, Ps cyt c5, Ps cyt c551, Mn cyt c552, and Ac paz strains, were reacted under the same conditions as described above in the presence of Fe(II)EDTA-NO, and the amounts of intermediate nitrous oxide (¹⁵N₂O) and final nitrogen (¹⁵N₂) product were measured.

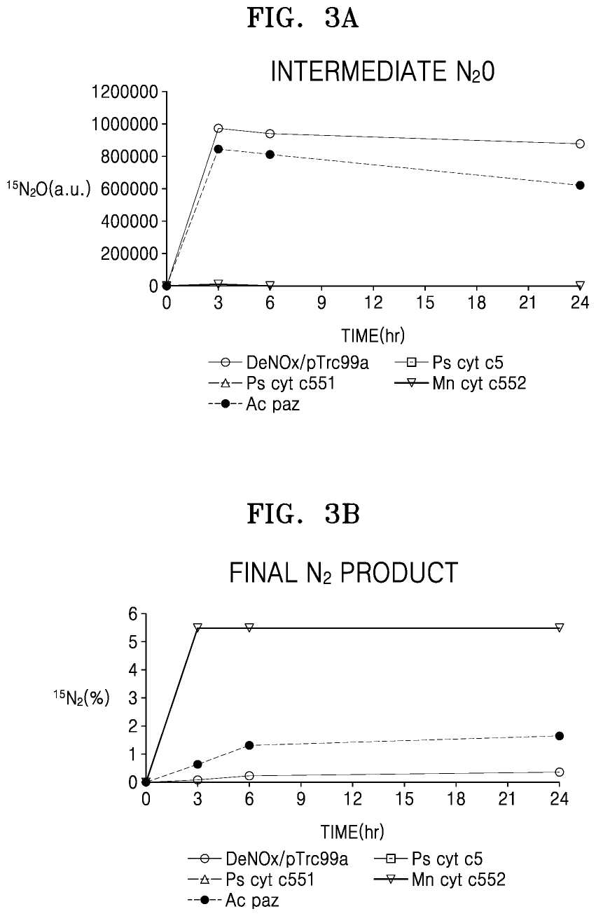

FIG. 3A is a diagram showing the concentration of intermediate nitrous oxide over time when a recombinant DeNOx E. coli strain including a genetic modification that introduces the expression of Ps cyt c5, Ps cyt c551, Mn cyt c552 or Ac paz was reacted in the presence of Fe(II)EDTA-¹⁵NO. DeNOx/pTrc99a is a control strain with an empty vector.

FIG. 3B is a diagram showing the concentration of the final product nitrogen over time when a recombinant DeNOx E. coli strain including a genetic modification that introduces the expression of Ps cyt c5, Ps cyt c551, Mn cyt c552 or Ac paz was reacted in the presence of Fe(II)EDTA-¹⁵NO.

As shown in FIGS. 3A and 3B, compared to the control strain DeNOx/pTrc99a, the concentration of intermediate nitrous oxide in the strains expressing Ps cyt c5, Ps cyt c551 or Mn cyt c552 was remarkably reduced, or was not measurable, from the beginning of the reaction and throughout the 24 hr reaction timespan, and the level of nitrogen produced in the strains expressing Ps cyt c5, Ps cyt c551 or Mn cyt c552 was remarkably increased within about 3 hr from the beginning of the reaction. The results indicate that the electron flow to the NosZ nitrous oxide reductase was strengthened by the introduced Ps cyt c5, Ps cyt c551 or Mn cyt c552 electron-transfer protein gene, thereby accelerating the conversion of nitrous oxide to nitrogen by the NosZ enzyme in the recombinant microorganism. The concentration of intermediate nitrous oxide, and the final product nitrogen over time corresponding to FIG. 3A, and FIG. 3B are shown in Table 3 and Table 4, respectively.

TABLE 3

| Recombinant | Intermediate ¹⁵N₂O (a.u.) | | |
|---|---|---|---|
| DeNOx E. coli | 3 h | 6 h | 24 h |
| DeNOx/pTrc99a | 976396 ± 33263 | 942770 ± 3643 | 880877 ± 83208 |
| Ps cyt c5 | 13856 ± 2761 | 0 ± 0 | 0 ± 0 |
| Ps cyt c551 | 15659 ± 851 | 0 ± 0 | 0 ± 0 |
| Mn cyt c552 | 12856 ± 2761 | 0 ± 0 | 0 ± 0 |
| Ac paz | 850229 ± 29694 | 813881 ± 44062 | 626317 ± 10945 |

TABLE 4

| Recombinant | Final product ¹⁵N₂ (%) | | |
|---|---|---|---|
| DeNOx E. coli | 3 h | 6 h | 24 h |
| DeNOx/pTrc99a | 0.09 ± 0.02 | 0.24 ± 0.00 | 0.37 ± 0.01 |
| Ps cyt c5 | 5.49 ± 0.08 | 5.51 ± 0.09 | 5.54 ± 0.09 |
| Ps cyt c551 | 5.48 ± 0.05 | 5.51 ± 0.02 | 5.51 ± 0.04 |
| Mn cyt c552 | 5.49 ± 0.08 | 5.48 ± 0.09 | 5.50 ± 0.09 |
| Ac paz | 0.64 ± 0.07 | 1.31 ± 0.00 | 1.65 ± 0.04 |

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the specification and the claims.

What is claimed is:

1. A recombinant microorganism of the genus Escherichia, comprising:
a genetic modification that increases expression of a gene encoding an electron-transfer protein; and
a genetic modification that increases expression of a nosZ gene encoding nitrous oxide reductase NosZ, a nosR gene encoding NosR, a nosD gene encoding NosD, a nosF gene encoding NosF, a nosY gene encoding NosY, and an apbE gene encoding ApbE;
wherein the nosZ gene, the nosR gene, the nosD gene, the nosF gene, the nosY gene and the apbE gene are derived from a microorganism of the genus Pseudomonas or Paracoccus.

2. The recombinant microorganism of claim 1, wherein the electron-transfer protein comprises c-type cytochrome, pseudoazurin, or amicyanin, or a combination thereof.

3. The recombinant microorganism of claim 1, wherein the electron-transfer protein is cytochrome c5 derived from Pseudomonas stutzeri, cytochrome c derived from Pseudomonas stutzeri, cytochrome c551 derived from Pseudomonas stutzeri, cytochrome c550 derived from Pseudomonas stutzeri, cytochrome c550 derived from Pseudomonas aeruginosa, cytochrome c552 derived from Marinobacter nauticus, pseudoazurin derived from Achromobacter cycloclastes, pseudoazurin derived from Sinorhizobium meliloti, pseudoazurin derived from Paracoccus versutus, or amicyanin derived from Paracoccus versutus, or any combination thereof.

4. The recombinant microorganism of claim 1, wherein the electron-transfer protein has a sequence identity of at least about 75%, 80%, 85%, 90%, 95% or 98% to the amino acid sequence of SEQ ID NO: 1, 3, 5, 7, 9, 11, 13, 15, 17, or 19.

5. The recombinant microorganism of claim 1, further comprising:
a genetic modification that reduces expression of one or more genes of a fur gene, an nsrR gene, and a lac gene; and
a genetic modification that increases expression of a ccm ABCDEFGH operon.

6. The recombinant microorganism of claim 1, further comprising a genetic modification that increases expression of a nosL gene encoding NosL.

7. The recombinant microorganism of claim 6, wherein the NosZ, the NosR, the NosL, the NosD, the NosF, the NosY, and the ApbE have a sequence identity of at least about 75%, 80%, 85%, 90%, 95% or 98% to the amino acid sequence of SEQ ID NO: 28, 30, 32, 34, 36, 38, or 40, respectively.

8. The recombinant microorganism of claim 6, wherein one or more of the nosZ gene, the nosR gene, the nosL gene, the nosD gene, the nosF gene, the nosY gene, and the apbE gene are integrated into a chromosome of the recombinant microorganism.

9. The recombinant microorganism of claim 6, wherein the nosZ gene, the nosR gene, and the nosL gene are contained in one operon, the nosD gene and the nosY gene are contained in one operon, and the apbE gene and the nosF gene are contained in one operon.

10. A composition for use in decreasing a concentration of nitrogen oxide in a sample, the composition comprising a recombinant microorganism comprising:
a genetic modification that increases expression of a gene encoding an electron-transfer protein; and
a genetic modification that increases expression of a nosZ gene encoding nitrous oxide reductase NosZ, a nosR gene encoding NosR, a nosD gene encoding NosD, a nosF gene encoding NosF, a nosY gene encoding NosY, and an apbE gene encoding ApbE;
wherein the nosZ gene, the nosR gene, the nosD gene, the nosF gene, the nosY gene and the apbE gene are derived from a microorganism of the genus Pseudomonas or Paracoccus.

11. The composition of claim 10, wherein the nitrogen oxide is in the form of:
nitric oxide (NO);
Fe(II)(L)-NO, wherein L is a chelating agent, and L, Fe²⁺, and nitric oxide (NO) form a chelating complex; or
nitrous oxide (N₂O).

12. The composition of claim 11, wherein the chelating agent L is ethylenediamine, diethylenetriamine, triethylenetetramine, hexamethylenetetramine, N-(2-hydroxyethyl)ethylenediamine-triacetic acid (HEDTA), ethylenediamine-tetraacetic acid (EDTA), iminodiacetic acid, nitrilo-triacetic acid (NTA), or diethylenetriamine pentaacetic acid (DTPA).

13. The composition of claim 10, wherein the electron-transfer protein comprises c-type cytochrome, pseudoazurin, or amicyanin, or any combination thereof.

14. The composition of claim 10, wherein the electron-transfer protein is cytochrome c5 derived from Pseudomonas stutzeri, cytochrome c derived from Pseudomonas stutzeri, cytochrome c551 derived from Pseudomonas stutzeri, cytochrome c550 derived from Pseudomonas stutzeri, cytochrome c550 derived from Pseudomonas aeruginosa, cytochrome c552 derived from Marinobacter nauticus, pseudoazurin derived from Achromobacter cycloclastes, pseudoazurin derived from Sinorhizobium meliloti, pseudoazurin derived from Paracoccus versutus, or amicyanin derived from Paracoccus versutus, or any combination thereof.

15. The composition of claim 10, wherein the electron-transfer protein has a sequence identity of at least about 75%, 80%, 85%, 90%, 95% or 98% to the amino acid sequence of SEQ ID NO: 1, 3, 5, 7, 9, 11, 13, 15, 17, or 19.

16. The composition of claim 10, wherein the recombinant microorganism further comprises:
a genetic modification that reduces expression of one or more genes of a fur gene, an nsrR gene, and a lac gene; and
a genetic modification that increases expression of a ccm ABCDEFGH operon.

17. A method of decreasing a concentration of nitrogen oxide in a sample, the method comprising decreasing a concentration of nitrogen oxide in a sample by contacting a recombinant microorganism of the genus Escherichia with a sample containing nitrogen oxide, wherein the recombinant microorganism comprises:
a genetic modification that increases expression of a gene encoding an electron-transfer protein; and
a genetic modification that increases expression of a nosZ gene encoding nitrous oxide reductase NosZ, a nosR gene encoding NosR, a nosD gene encoding NosD, a nosF gene encoding NosF, a nosY gene encoding NosY, and an apbE gene encoding ApbE;
wherein the nosZ gene, the nosR gene, the nosD gene, the nosF gene, the nosY gene and the apbE gene are derived from a microorganism of the genus Pseudomonas or Paracoccus.

18. The method of claim 17, wherein the nitrogen oxide is in the form of:
nitric oxide (NO); Fe(II)(L)-NO, wherein L is a chelating agent, and L, Fe²⁺, and nitric oxide (NO) form a chelating complex; or
nitrous oxide (N₂O).

19. The method of claim 18, wherein the chelating agent L is ethylenediamine, diethylenetriamine, triethylenetetramine, hexamethylenetetramine, N-(2-hydroxyethyl)ethylenediamine-triacetic acid (HEDTA), ethylenediamine-tetraacetic acid (EDTA), iminodiacetic acid, nitrilo-triacetic acid (NTA), or diethylenetriamine pentaacetic acid (DTPA).

20. The method of claim 17, wherein the electron-transfer protein comprises c-type cytochrome, pseudoazurin, or amicyanin, or any combination thereof.

21. The method of claim 17, wherein the electron-transfer protein is cytochrome c5 derived from Pseudomonas stutzeri, cytochrome c derived from Pseudomonas stutzeri, cytochrome c551 derived from Pseudomonas stutzeri, cytochrome c550 derived from Pseudomonas stutzeri, cytochrome c550 derived from Pseudomonas aeruginosa, cytochrome c552 derived from Marinobacter nauticus, pseudoazurin derived from Achromobacter cycloclastes, pseudoazurin derived from Sinorhizobium meliloti, pseudoazurin derived from Paracoccus versutus, or amicyanin derived from Paracoccus versutus, or any combination thereof.

22. The method of claim 17, wherein the recombinant microorganism further comprises:
a genetic modification that reduces expression of one or more genes of a fur gene, an nsrR gene, and a lac gene; and
a genetic modification that increases expression of a ccm ABCDEFGH operon.

23. The method of claim 17, wherein the recombinant microorganism further comprises a genetic modification that increases expression of a nosL gene encoding NosL.

24. The method of claim 17, wherein the contacting is performed in a closed container under anaerobic conditions.

25. The method of claim 17, wherein the contacting comprises culturing or incubating the recombinant microorganism in the presence of the sample containing nitrogen oxide.


SEQUENCE LISTING (95 sequences)

SEQ ID NO: 1 - protein, 141 aa, Pseudomonas stutzeri
MNLIKKILVAQTAVVALWAMSAQAATDEAIAERLKPVGEVCIQGEECAAAGAGAVAAAGGAARSGEEVYGKFCTACHGSGLLNAPKTGDSAAWKGRADAAGGLDGLLKHAISGINAMPPKGTCADCSDDELKSAIQHMSGL

SEQ ID NO: 2 - DNA, 426 bp, Pseudomonas stutzeri
  source 1..426: mol_type = genomic DNA
atgaacttaattaagaagatccttgtcgcgcagacagctgtagttgccctttgggccatgtccgctcaagcggcgacggatgaagctattgctgaacgtttgaagccggtaggcgaagtatgtattcaaggggaggaatgtgcagcagctggtgctggtgcagtcgctgctgctggtggagccgcacgttcaggggaggaagtatatggtaagttttgcacagcatgccatggttcgggccttctgaatgctccgaaaaccggggatagtgcagcttggaagggacgtgccgatgcagctgggggtttggatggccttttgaagcacgcaatttcggggattaacgccatgccgcctaaggggacatgcgcggattgctctgatgacgagctgaagtcggcaatccagcatatgagtggcttataa

SEQ ID NO: 3 - protein, 103 aa, Pseudomonas stutzeri
MGLALASLLLIPAALAAAPDAERQAQLEHLLVQDCGSCHGLRMTGGLGPAITRDALAGKPRDSLIATVTHGRPGTAMPGWNALLDEQDIVWLVDLLLEGYPKP

SEQ ID NO: 4 - DNA, 312 bp, Pseudomonas stutzeri
  source 1..312: mol_type = genomic DNA
atggggctggctcttgcatccctgttgcttattccggcggccttggccgcagctccagatgctgagcgccaggcccaattagagcatctgttagtccaggactgtggatcatgtcacgggttacgcatgactggaggattgggacctgccattacacgcgacgctttggccgggaaaccgcgtgatagcttgattgccacggttacgcatgggcgtcctgggactgccatgccaggatggaacgctttacttgacgaacaggacatcgtctggttagtagacctgttattagaaggttatccgaagccataa

SEQ ID NO: 5 - protein, 104 aa, Pseudomonas stutzeri
MKKILVPLFALSGALMLQPAIAQDGEALFKSKPCAACHSIDAKLVGPAFKEVAAKYAGQEGAADLLAGHIKNGSQGVWGPIPMPPNAVTEEEAKILAEWVLSQK

SEQ ID NO: 6 - DNA, 315 bp, Pseudomonas stutzeri
  source 1..315: mol_type = genomic DNA
atgaaaaaaattctggtgccgctgtttgcgctgagcggcgcgctgatgctgcagccggcgattgcgcaagatggcgaagcgctgtttaaaagcaaaccgtgcgcggcgtgccatagcattgatgcgaaactggtgggcccggcgtttaaagaagtggcggcgaaatatgcgggccaagaaggcgcggcggatctgctggcgggccatattaaaaacggcagccaaggcgtgtggggcccgattccgatgccgccgaacgcggtgaccgaagaagaagcgaaaattctggcggaatgggtgctgagtcagaaataa

SEQ ID NO: 7 - protein, 145 aa, Pseudomonas stutzeri
MNNKNPMRVLLGAGLLAMSGLALAHGDVVPHAVETKGLPELGEEWVDENPYREPGEHHDLAVEIGSSAYNQNCARCHGLEAISGGIAPDLRELEADISGDEWFKERVINGAVRDGAVYMPRMADTLSQEALWAIRTYIENEAAKQ

SEQ ID NO: 8 - DNA, 438 bp, Pseudomonas stutzeri
  source 1..438: mol_type = genomic DNA
atgaataacaaaaatccgatgcgtgtactgctgggagctgggttattagcgatgtctggtctggccttggcgcatggggacgtagtaccacacgctgttgaaacaaagggcttaccggagcttggcgaggagtgggtcgatgaaaacccatacagagaaccaggcgaacatcacgacttagcggtagagattggctcttcagcctataaccaaaattgtgcgcgctgccacggtcttgaagctatcagcggtgggattgcgcctgatttgcgcgagttagaggcagacatttctggcgacgaatggttcaaagagcgcgtaatcaatggcgctgtgcgtgatggagcagtctatatgccacgtatggccgacaccttatcgcaggaggcgctgtgggcgatccgcacctacattgagaacgaggctgcaaagcagtaa

SEQ ID NO: 9 - protein, 145 aa, Pseudomonas aeruginosa
MNKNNVLRGLLVLAGLSLSSLALAHGDVTPQAVDTKGLEPLGKEWRDTNPYRKPYAKHDLAVEIGASAYNQNCARCHGLEAKSGGIAPDLRLLETGAEGDEWFKERVINGAVRDGAVYMPKMADFISQEGLWAIRSYLESVHVDE

SEQ ID NO: 10 - DNA, 438 bp, Pseudomonas aeruginosa
  source 1..438: mol_type = genomic DNA
atgaacaaaaacaacgtgctgcgcggcctgctggtgctggcgggcctgagcctgagcagcctggcgctggcgcatggcgatgtgaccccgcaagcggtggataccaaaggcctggaaccgctgggcaaagaatggcgcgataccaacccgtatcgcaaaccgtatgcgaaacatgatctggcggtggaaattggcgcgagcgcgtataatcagaactgcgcgcgctgccatggcctggaagcgaaaagcggcggcattgcgccggatctgcgcctgctggaaaccggcgcggaaggcgatgaatggtttaaagaacgcgtgattaacggcgcggtgcgcgatggcgcggtgtatatgccgaaaatggcggattttattagccaagaaggcctgtgggcgattcgcagctatctggaaagcgtgcatgtggatgaataa

SEQ ID NO: 11 - protein, 112 aa, Marinobacter nautica
MKLKHLAVAGLFAAGAMVPVASMAAGDIEAGKAKAAVCAACHGQNGISQVPIYPNLAGQKEQYLVAALKAYKAGQRQGGQAPVMQGQATALSDADIANLAAYYASLPADGGK

SEQ ID NO: 12 - DNA, 339 bp, Marinobacter nautica
  source 1..339: mol_type = genomic DNA
atgaaattgaaacatttagcggttgccggcttattcgcggcgggggccatggtgccggtggcgagcatggcggcgggcgatattgaagcgggcaaagcgaaagcggcggtgtgcgcggcgtgccatggtcagaacggcattagccaagtgccgatttatcccaatctggcgggtcagaaagaacagtatctggtggcggcgctgaaggcgtacaaggcgggacagcgccaaggtggccaagcgcctgtgatgcaaggccaagcgaccgcgctgagcgatgcggatattgccaacctggcagcgtattatgcgagcctgccggcggatggcggcaaataa

SEQ ID NO: 13 - protein, 152 aa, Achromobacter cycloclastes
MEKTMLNAIKSGFGIAIAAMLVAAPAAAADFEVHMLNKGKDGAMVFEPASLKVAPGDTVTFIPTDKGHNVETIKGMIPDGAEAFKSKINENYKVTFTAPGVYGVKCTPHYGMGMVGVVQVGDAPANLEAVKGAKNPKKAQERLDAALAALGN

SEQ ID NO: 14 - DNA, 459 bp, Achromobacter cycloclastes
  source 1..459: mol_type = genomic DNA
atggaaaaaaccatgctgaacgcgattaaaagcggctttggcattgcgattgcggcgatgctggtggcggcgccggcggcggcggcggattttgaagtgcatatgctgaacaaaggcaaagatggcgcgatggtgtttgaaccggcgagcctgaaagtggcgccgggcgataccgtgacctttattccgaccgataaaggccataacgtggaaaccattaaaggcatgattccggatggcgcggaagcgtttaaaagcaaaattaacgaaaactataaagtgacctttaccgcgccgggcgtgtatggcgtgaaatgcaccccgcattatggcatgggcatggtgggcgtggtgcaagtgggcgatgcgccggcgaacctggaagcggtgaaaggcgcgaaaaacccgaaaaaagcgcaagaacgcctggatgcggcgctggcggcgctgggcaactaa

SEQ ID NO: 15 - protein, 147 aa, Sinorhizobium meliloti
MRIIAKGMAVAAVLAAFTGSAFAADFEVRMLNKGSEGVMVFEPAFVKVNPGDSVTFVPTDKGHNVETIKDMIPDGASAFKSKMNETYKVTFDVPGVYGVKCTPHVGMGMVAAVVVGDAPANVEKVKAVKLPKKARERLDAALAVALQ

SEQ ID NO: 16 - DNA, 444 bp, Sinorhizobium meliloti
  source 1..444: mol_type = genomic DNA
atgcgcattattgcgaaaggcatggcggtagctgcggtgctggcggcgtttaccggcagcgcgtttgcggcggattttgaagtgcgcatgctgaacaaaggcagcgaaggcgtgatggtgtttgaaccggcgtttgtgaaagtgaacccgggcgatagcgtgacctttgtgccgaccgataaaggccataacgtggaaaccattaaagatatgattccggatggcgcgagcgcgtttaaaagcaaaatgaacgaaacctataaagtgacctttgatgtgccgggcgtgtatggcgtgaaatgcaccccgcatgtgggcatgggcatggtcgcggctgtggtagttggcgatgcgccggcgaacgtggaaaaagtgaaagcggtgaaactgccgaaaaaagcgcgcgaacgcctggatgcggcgctggcggtggcgctgcagtaa

SEQ ID NO: 17 - protein, 145 aa, Paracoccus versutus
MFHNSLAAAAAALLALAAPAFAATHEVHMLNKGESGAMVFEPAFVRAEPGDVINFVPTDKSHNVEAIKEILPEGVETFKSKINESYTLTVTEPGLYGVKCTPHFGMGMVGLVQVGEAPDNLDAAKTAKMPKKARERMDAELAQVN

SEQ ID NO: 18 - DNA, 438 bp, Paracoccus versutus
  source 1..438: mol_type = genomic DNA
atgtttcacaacagcctggcggcggctgcggcggcgcttctggcattagcggcgcctgcgttcgcggcgacccatgaagtgcatatgctgaacaaaggcgaaagcggcgcgatggtgtttgaaccggcgtttgtgcgcgcggaaccgggcgatgtgattaactttgtgccgaccgataaaagccataacgtggaagcgattaaagaaattctgccggaaggcgtggaaacctttaaaagcaaaattaacgaaagctataccctgaccgtgaccgaaccgggcctgtatggcgtgaaatgcaccccgcattttggcatgggcatggtgggcctggtgcaagtgggcgaagcgccggataacctggatgcggcgaaaaccgcgaaaatgccgaaaaaagcgcgcgaacgcatggatgcggaactggcgcaagtgaactaa

SEQ ID NO: 19 - protein, 132 aa, Paracoccus versutus
MISAKTLRPAIAAIALFAIGATGAWAQDKITVTSEKPVAAADVPADAVVVGIEKMKYLTPEVTIKAGETVYWVNGEVMPHNVAFKKGIVGEDAFRGEMMTKDQAYAITFNEAGSYDYFCTPHPFMRGKVIVE

SEQ ID NO: 20 - DNA, 399 bp, Paracoccus versutus
  source 1..399: mol_type = genomic DNA
atgattagcgcgaaaaccctgcgcccggcgattgcggcgattgcgctgtttgcgattggcgcgaccggcgcgtgggcgcaagataaaattaccgtgacgagcgaaaaaccggtggcggcggcggatgtgccggcggatgcggtggtggtgggcattgaaaaaatgaaatatctgaccccggaagtgaccattaaagcgggcgaaaccgtgtattgggtgaacggcgaagtgatgccgcataacgtggcgtttaaaaaaggcattgtgggcgaagatgcgtttcgcggcgaaatgatgaccaaagatcaagcgtatgcgattacctttaacgaagcgggcagctatgattatttttgcaccccgcatccgtttatgcgcggcaaagtgattgtggaataa

SEQ ID NO: 21 - protein, 148 aa, Escherichia coli
MTDNNTALKKAGLKVTLPRLKILEVLQEPDNHHVSAEDLYKRLIDMGEEIGLATVYRVLNQFDDAGIVTRHNFEGGKSVFELTQQHHHDHLICLDCGKVIEFSDDSIEARQREIAAKHGIRLTNHSLYLYGHCAEGDCREDEHAHEGK

SEQ ID NO: 22 - DNA, 447 bp, Escherichia coli
  source 1..447: mol_type = genomic DNA
atgactgataacaataccgccctaaagaaagctggcctgaaagtaacgcttcctcgtttaaaaatcctggaagttcttcaggagccggacaaccatcacgtcagtgcggaagatttatacaaacgtctgatcgatatgggtgaagaaattggtctggctacggtatatcgcgtactgaaccagtttgacgacgctggtatcgtcacccgccacaattttgaaggcggtaaatccgtatttgaactgacacagcaacatcaccacgatcacctgatctgcctcgactgcggcaaggttatcgaatttagtgatgattccatcgaagcgcgtcagcgtgaaattgccgcaaaacatggcattcgcctgactaaccacagtctctatctttacggtcactgtgccgaaggcgattgccgcgaagatgaacatgcgcacgaaggcaaataa

SEQ ID NO: 23 - protein, 141 aa, Escherichia coli
MQLTSFTDYGLRALIYMASLPEGRMTSISEVTDVYGVSRNHMVKIINQLSRAGYVTAVRGKNGGIRLGKPASAIRIGDVVRELEPLSLVNCSSEFCHITPACRLKQALSKAVQSFLTELDNYTLADLVEENQPLYKLLLVE

SEQ ID NO: 24 - DNA, 426 bp, Escherichia coli
  source 1..426: mol_type = genomic DNA
gtgcagttaacgagtttcactgattacggattacgtgcgctgatctacatggcgtcattgccagaagggcggatgaccagtatttctgaagtgactgacgtctacggcgtctcccgtaatcatatggtcaaaataatcaatcaacttagtcgtgccggctacgtgactgctgttcgtggaaaaaatggcggcattcgcctgggtaaaccggcgagtgcgatacgtattggtgatgtggtgcgcgagctggagcccttatcgctggtgaattgcagcagtgagttttgccacattacacctgcctgtaggttgaaacaggcactttctaaggccgtgcaaagttttcttacggaactggataactacacgcttgccgatttggttgaagagaatcaaccgctttataaattattgctggtggagtga

SEQ ID NO: 25 - protein, 360 aa, Escherichia coli
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 26 - DNA, 1083 bp, Escherichia coli
  source 1..1083: mol_type = genomic DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 27 - DNA, 100 bp, Escherichia coli
  source 1..100: mol_type = genomic DNA
ggtgaaagatgggcaaacctgtattgattgccataaagggatagcgcacaagctgcccgatatgcgtgaagtcgagccaggtttttaacagggttattgc

SEQ ID NO: 28 - protein, 638 aa, Pseudomonas stutzeri
MSDNDSKNTPQTLEKNGLSRRGFLGASALTGAAVAATALGGAVMSRESWAAAVKDAQSKIHVGPGELDEYYGFWSGGHQGEVRVLGVPSMRELMRIPVFNVDSATGWGLTNESRAIMGDSAKFLNGDCHHPHISMTNGKYDGKYLFINDKANSRVARIRLDIMKCDKMITVPNVQAIHGLRLQKVPHTKYVFANAEFIIPHPNDGKVFDLQDENSYTMYNAIDAESMEMAFQVIVDGNLDNTDADYTGRFAAATCYNSEKAFDLGGMMRNERDWVVVFDIHAIEKAVKAGKFITLGDSKVPVVDGRKKDGKNSEFTRYVPVPKNPHGLNTSSDGKYFIANGKLSPTCSMIAIDMLPDLFAGKLKDERDVVVGEPELGLGPLHTTFDGRGNAYTTLFIDSQVVKWNMEEARRAYKGEKVNYIKQKLDVHYQPGHLHASLCETSEADGKWLVALSKFSKDRFLPTGPLHPENDQLIDISGDEMKLVHDGPTFAEPHDCIMARRDQIKTKKIWDRNDPFFAPTVKMAEKDGINLTTDNKVIRDGNKVRVYMTSMAPAYGITDFTVKQGDEVTVVVTNIDQIEDVSHGFVVVNHGVSMEISPQQTSSITFVADKPGLHWYYCSWFCHALHMEMVGRMIVEPA

SEQ ID NO: 29 - DNA, 1917 bp, Pseudomonas stutzeri
  source 1..1917: mol_type = genomic DNA
atgtccgacaatgacagcaaaaatacgccacagactctggagaagaacgggttgtcgcggcgcggctttttaggggcgagcgcattgacaggtgctgctgtagcagcgacagctctcggcggtgctgtcatgtcgcgcgagtcgtgggctgcagccgtgaaagatgctcaatcgaaaatccatgttgggccaggggaactcgatgaatactacggcttctggtcgggtgggcatcagggggaagtccgtgttctgggcgtccctagtatgcgtgaactcatgcggattcctgtattcaacgtcgactcagccacaggttggggcctcacaaatgaaagccgtgccattatgggtgattccgcaaagttcctcaatggcgattgtcatcatccacacatctcgatgactaacggcaaatatgatggtaaatacttatttattaatgacaaggccaatagtcgggttgcacggattcgcctcgatattatgaagtgcgataaaatgattacagtcccaaacgtacaggctatccatggcctccggcttcaaaaggtaccgcatacaaagtacgtcttcgcgaacgcggaattcatcattcctcatccaaatgacggcaaggtcttcgatttacaagacgagaattcctatacgatgtacaatgcgattgacgcagaaagcatggaaatggcctttcaggtgattgtcgatgggaacttagacaacacagacgccgactatactggtcggttcgcagcagctacatgttacaactctgagaaagcctttgaccttggtggtatgatgcggaatgagcgtgactgggtcgttgtattcgacattcatgctattgaaaaggcagttaaagccggcaagtttattacgttaggcgattctaaagtgccagtggtagacggccgcaagaaggatggtaagaactccgagttcacgcgctacgtgcctgtgccaaaaaacccacatggtttaaatacttcgagtgacggtaagtatttcatcgcaaacgggaaactcagcccgacatgctctatgattgcgattgatatgcttccggacctgttcgctgggaaactcaaggatgaacgtgatgtggttgttggtgagccagaacttgggctgggtcctctgcacactacgtttgacgggcgcgggaatgcctacacaacactgtttatcgactcccaagttgtgaagtggaacatggaagaagcgcgccgtgcttacaaaggggagaaggttaattatattaaacagaagcttgacgtccattatcaaccggggcatctccatgcttcgttgtgtgagacgtcagaagcagatggtaaatggctggtggctttatctaaattttcgaaggaccgtttcctgccaactggtcctctccacccggagaacgaccagttaatcgacatcagcggggacgaaatgaagctggtgcacgacggccctacgtttgcggaacctcacgactgtattatggcacgtcgggaccaaattaaaacgaagaaaatctgggatcgcaatgacccgttttttgcacctacggtcaagatggcagagaaggatggtatcaacttgactaccgataacaaggtgatccgggatgggaataaagttcgcgtgtacatgactagtatggcacctgcctacgggattaccgatttcaccgtgaaacaaggtgacgaagttaccgttgtggttacaaatatcgatcaaattgaggacgtcagtcacggttttgtcgtagtcaatcacggtgtatcaatggaaatctcaccacaacagacatcttccattacgtttgtcgccgacaaaccggggttacattggtactattgtagctggttctgccatgcccttcatatggagatggtaggtcgcatgattgtagaaccagcatga

SEQ ID NO: 30 - protein, 726 aa, Pseudomonas stutzeri
MKPLGFAVVGTSRRWVAGFAILLALMFCVSVAQAKEYEAEQQRIELLFPKVTEISEPEGEYQVRTLADGVGTIYGYAFQSINVTDMPAYSGKPINMQILLDPKGVIVDAYMLEHHEPIVLIGIPEQKVHDFNANYSGIHVDQRVVVGRSSDKSAVTVDAVTGATVTVMVINEIVMRAAHTVAVDLGLVEAGATARPKPALVREDVFQPTSWTELVGNGAIRRMHLTRGQVDDAFKGTEAEGVDVAAAEQRDETFIDLYATHLNPPTIGRNLLGERQYADLMANLKPGEHAFAVLANGEYSFKGSGYVRGGIFDRVQLRQFGDTISFRDLDFIRLSDVYAEGMPEFFEMAIFTAREQYRFDPGSPWNLELLVRRQVGPVESIFTSFEMPYVMPEEYIERVPLTAEELAAIEEANRPLWVNIWYQKSFQVGVILVALALLTVILFLQDKFTQHPNFLKRLRHGYLVFTVVFIGWYALGQLSVVNVLTFVHALVQDFRWELFLTDPVIFILWVFTAASILLWGRGVFCGWLCPFGALQELINEAARKLKIPQYDLPFGVHERLWAIKYIVLLVLFGISLESMMMAEKAAEIEPFKTAITLKFDRQWWFVAYAVFLLVINIFTRKVYCRYVCPLGAGLAITGRFRLFDWLKRRKECGNPCQICANECEVQAIHPDGHINHNECHYCLDCQMTYHNENKCPPLIQKNKRKKRDKKAPVGAELIPVVQVVEP

SEQ ID NO: 31 - DNA, 2181 bp, Pseudomonas stutzeri
  source 1..2181: mol_type = genomic DNA
atgaagccgttaggtttcgccgtcgtaggcacatcccgccgctgggttgctggtttcgcaatcctgctcgccctgatgttttgtgttagtgtagcgcaggctaaagagtacgaagcagagcaacagcgcattgaattactctttcctaaagtgactgagatctcagaaccggagggcgaataccaagtgcgtacgcttgcagatggtgtgggtacgatctatgggtatgccttccagtccatcaatgtaaccgatatgccggcgtatagtggtaaaccgatcaatatgcagattctcctggacccaaagggggtaatcgtcgacgcttacatgctcgagcaccatgaacctattgtgctgatcggtattccggaacagaaagttcacgacttcaacgctaattacagtgggatccatgttgaccagcgcgttgtcgtaggtcgtagttccgataaatcggcagttaccgtggatgccgtcactggcgctaccgtcacagtaatggtcatcaacgagatcgtcatgcgcgcggctcacacggtcgcagtagatttgggtctcgttgaggctggtgccactgcccgcccgaagccggcattagtgcgcgaagacgttttccagccaacatcgtggaccgagcttgtgggcaatggggctattcgccgtatgcacttaacccgcggtcaagttgacgatgcctttaaaggtactgaagctgagggcgtggatgttgctgccgctgagcagcgcgacgaaacctttattgacttatatgcgacccatcttaacccacctacaatcggtcgtaacttacttggggaacgccagtatgcggatctgatggcaaatctgaaaccaggcgagcacgctttcgccgttttagcgaatggcgagtacagcttcaaagggtccggctatgtgcgcggcgggatttttgatcgcgtgcaactccgtcagtttggggacactatctcttttcgtgatttggattttattcgtctttctgacgtatatgcggaaggtatgccagagtttttcgaaatggcaatttttactgcacgcgaacaataccgctttgacccagggtctccgtggaatctcgaattattggtacgccgtcaagtcggtccagtcgagtcaatctttacttcattcgaaatgccttacgtgatgcctgaagaatacatcgagcgcgtgccgcttaccgctgaagaacttgccgcaatcgaggaagcgaaccgccctctttgggtcaatatctggtatcagaaatcgtttcaagttggggtgatcctggtcgcccttgctctcttaacggtaattttgttccttcaagacaaatttacccagcatcctaacttccttaagcgcttgcgccatggctacctcgtattcaccgtcgtttttattggctggtatgcattaggccagttatctgtcgtgaacgtgcttacattcgttcacgcacttgtgcaggactttcgctgggagctgtttctcaccgaccctgtgatcttcatcctgtgggtcttcaccgctgccagcattcttctctgggggcgcggggtcttttgtgggtggctgtgtccatttggcgccctccaggaacttatcaatgaagccgcgcgcaagctgaagatcccgcagtatgatcttccgtttggcgttcatgagcgcctttgggccattaagtatatcgtactgctggtcttatttggtatctctttagagtcaatgatgatggcggaaaaggccgcagaaattgagccttttaaaactgcgatcacactgaaattcgaccgccaatggtggttcgtggcctatgccgttttcttgttggttatcaatattttcacccgcaaggtttattgccgttatgtttgtcctctcggtgccggcctcgctatcaccggtcgttttcgcttatttgattggcttaaacgtcgtaaagagtgtggcaacccgtgtcagatctgtgctaacgagtgcgaggtacaggcgatccatcctgatgggcacatcaaccacaatgagtgtcactattgcttggactgccaaatgacgtaccataatgaaaacaagtgtccaccgttgattcaaaagaataagcgtaaaaagcgcgacaaaaaagccccggtaggcgcagagttaatcccagtggtccaagtggttgagccttga

SEQ ID NO: 32 - protein, 192 aa, Pseudomonas stutzeri
MMNNLYRTGAQALLAMLLAFGLAACGDKQEVQQSLDPVAFHESDECHVCGMIIADFPGPKGQAVDKAGVKKFCSTAEMLGWYLQPENKLLDAKLYVHDMGKSEWAHPSDEHLIDARTAWYVAGTPLKGAMGVSLASFADEQQAQQLAAEHGGRVLRFEEIDHGVLQQGADMQHHDMHEQHMSETEHNDHAGH

SEQ ID NO: 33 - DNA, 579 bp, Pseudomonas stutzeri
  source 1..579: mol_type = genomic DNA
atgatgaataatttgtaccgcaccggtgctcaggcgttgcttgctatgcttttggcttttggtctcgcggcgtgtggcgacaagcaagaagtccagcaatccttggatccagtagcgttccatgagtccgatgaatgtcacgtgtgcggcatgattattgccgacttccctggtccaaaaggccaggcagttgataaggccggtgtcaagaagttttgcagcacagcagagatgcttgggtggtatctccagccagaaaataaattgctcgatgcgaaactttacgtacatgatatgggtaagtcagaatgggcacatccgagcgatgagcacctgatcgacgcacgcactgcctggtatgttgctggtacaccattaaagggtgcaatgggcgtctccttagcctctttcgccgacgaacaacaggcgcagcaactggcagcagaacacggcgggcgtgtactgcgcttcgaggagattgaccacggtgtgctccagcagggggccgatatgcagcaccacgatatgcacgaacaacacatgagcgaaacagaacataatgaccatgcaggccattga

SEQ ID NO: 34 - protein, 434 aa, Pseudomonas stutzeri
MRACYTLALTALMIPAGAALAEPQPLTSLPLQAQGENRWTLPAGEYIGQFIIDQPLALHCAPGAVIKAPGQGNVLTVRAANVTIEGCALRDWGRDLTAMNAAIFLEPTATGAQIRNNDLQGPGFGIWADRNRDLLVEGNRIEGDLGLRSQDRGNGIHLFSVRGARVIDNHVWNTRDGIYIDNSNGNSIERNLFEDLRYGVHYMFSHENRVIANVTRRTRTGYALMQSRKLTVIGNRSEHDQNYGILMNYITYSTLKDNFVTDVERGDTGGDSMISGGEGKALFIYNSLFNTIENNHFQRSDLGIHLTAGSEDNRISSNAFVGNAQQVKYVAIRTQEWSVDGRGNYWSDYLGWDRNEDGLGDIAYEPNDNVDRLLWMYPQVRLLMNSPSIEVLRWVQRAFPVIKSPGVQDSHPLMKPPTRGVTEEPMNTTQRPHS

SEQ ID NO: 35 - DNA, 1305 bp, Pseudomonas stutzeri
  source 1..1305: mol_type = genomic DNA
atgcgcgcttgctacactctcgcccttacggcgttaatgattccggccggggcggctcttgcggaaccacagccgcttacctccttgcctctgcaagcccagggcgaaaatcgctggacgctcccagcaggcgagtacattggccaattcatcattgaccaaccgctggcactccattgtgcacctggggcggttattaaagctccggggcagggtaatgtcttgacagtccgcgcagcaaacgtgacaatcgaggggtgcgcattgcgcgactggggccgcgacctcacggccatgaatgcagccatcttcttggaacctaccgccacgggcgcacagatccgcaataacgatttacaaggcccaggtttcggcatctgggccgaccgtaatcgtgatttgcttgtggaaggcaatcgtatcgaaggtgaccttggcttgcgttcccaggaccgcgggaatggcatccaccttttctctgtacgtggcgcacgcgtaattgacaaccatgtttggaacacacgcgatggcatctatattgacaactccaacggcaattctatcgagcgtaatctgtttgaggacttgcgctacggggtacactatatgttctcacacgaaaaccgcgttatcgcgaacgtcacccgccgcactcgcacaggctacgctttaatgcagagtcgcaaattgaccgttatcggcaatcgctccgaacacgatcaaaactatgggattttaatgaactacattacttatagcaccctcaaagataattttgtcacggatgtcgaacgcggtgatactggcggggattctatgatctcagggggtgaaggtaaagctctctttatctacaatagtctcttcaatacaattgaaaacaaccactttcaacgttccgatctcggcatccatctgacagccggctcagaggataatcgcatctcctccaacgcattcgtggggaatgctcagcaagtcaaatacgtggcaatccgtactcaagagtggagtgtggacggccgcggtaactactggtcggattaccttgggtgggatcgcaacgaagacgggcttggcgatattgcctatgaacctaacgacaatgtagaccgtcttctttggatgtatccacaggttcgcttattgatgaatagcccttcaatcgaggttcttcgttgggttcaacgtgcctttcctgtgatcaaaagccctggtgtccaggattcccatccgcttatgaaacctccaacgcgcggtgtgactgaggaacctatgaacacgactcagcgtcctcatagctaa

SEQ ID NO: 36 - protein, 308 aa, Pseudomonas stutzeri
MNAVEIQGVSQRYGNMTVLHDLNLNLGEGEVLGLFGHNGAGKTTSMKLILGLLQASEGRVQVLGRDPRTTDVRRHLGYLPENVTFYPQLTGRETLRHFARLKSTPLGQVDDLLEQVGLAHAADRRVKTYSKGMRQRLGLAQAVLGEPRLLLLDEPTVGLDPIATQELYLLIDRLRQTGTSVILCSHVLPGVEAHINRAAILAKGRLQAIGSLKQLRSEAGLPVRIRASGISQSEAWLERWANAGHSAQRLGEDGVEVIAINGHKLPLLRELLGESEPDDVEIHQPSLEDLYRYYMERAGDVQAAEGRV

SEQ ID NO: 37 - DNA, 927 bp, Pseudomonas stutzeri
  source 1..927: mol_type = genomic DNA
atgaacgctgtggaaattcaaggggtttctcagcgctatgggaatatgactgtactgcatgatttgaacttgaacttaggggaaggtgaagtgcttgggttattcggtcacaatggcgctgggaagacaacatcaatgaaacttatcctgggcctcctgcaagcatccgagggtcgtgtccaggtgctgggccgtgacccacgtaccactgatgtgcgtcgccacctcggttacttacctgaaaatgtcacgttttatcctcaacttacagggcgcgagactttacgtcacttcgcccgtctgaagtccacccctctgggccaagtggacgatctgctggagcaagttggtctcgctcacgctgcagaccgccgcgtgaaaacctactcaaaaggtatgcgccaacgcttgggcttagcgcaggcggtactgggtgagcctcgccttctgctgctggatgaaccaactgttgggttggaccctattgcgacgcaagagctctatctcttaattgatcgcctccgtcaaactggtacgtcggttatcctgtgttcacacgttcttccaggtgtagaggctcacattaatcgcgcagcgatccttgctaagggtcgcctccaagccatcgggtcactcaaacaattacgcagcgaagccgggttacctgtccgtattcgtgcctcgggtatttcccagagtgaggcctggttagagcgttgggcgaatgcggggcattcggcgcaacgtttaggcgaggatggcgttgaagtgattgcgatcaatgggcacaaactgccattgctccgtgagttattgggggaatcagaaccagacgacgtggaaattcaccaaccgtcgctggaagatctgtatcgctactatatggaacgtgcaggtgacgtgcaggccgcagaagggcgcgtataa

SEQ ID NO: 38 - protein, 275 aa, Pseudomonas stutzeri
MNQVWNIARKELSDGLRNRWLLAISLLFAVLAVGIAWLGAAASGQVGFTSIPATIASLASLATFLMPLIALLLAYDAIVGEDEGGTLMLLLTYPLGRGEILLGKFVGHGLILALAVLIGFGGAALAIALLVEDIELGLLLWAFGRFMVSSTLLGWVFLALAYVLSGKVNEKSSAAGLALGIWFLFVLVFDLVLLALLVLSEGKFSPELLPWLLLLNPTDIYRLINLSGFEGGGAMGVLSLGSDLPVPNAVLWLCLLAWAGASLLLAYGIFRRRLA

SEQ ID NO: 39 - DNA, 828 bp, Pseudomonas stutzeri
  source 1..828: mol_type = genomic DNA
atgaaccaagtgtggaacattgcgcgtaaagagttgtctgatggtctccgtaaccgctggttacttgctatttcattactctttgccgtattggctgtcggcatcgcttggctcggcgccgctgcgtctggtcaagttggtttcacgtctatcccagcgactatcgcgtccttagcgtcactggcaactttcctgatgcctttaattgcacttcttctcgcttatgatgccattgtcggtgaagatgagggggggactctcatgttattactgacctacccgttgggccgtggggaaatcttactgggcaagttcgtggggcatggcttaatcttggctttagctgtactcatcgggtttggcggggcggctttagctattgccctcttggtcgaggacattgaacttgggctgctgctctgggcattcggccgcttcatggtgagtagtacgctcctcgggtgggtatttttggccctcgcttacgttctttccggcaaagtcaatgagaagtcttctgcggcaggtctggcgctggggatttggttcttgtttgtacttgtgttcgatcttgtccttttagctctgctggtattgtccgaagggaagtttagtcctgaattattgccttggcttttgttattgaatcctaccgacatctatcgtcttattaatttgtccggctttgaaggtggtggggcgatgggggtattgtcattggggagtgaccttcctgtaccaaatgcggtcttatggctttgcctcctggcttgggccggcgcgtcattgcttttggcgtatggcattttccgtcgtcgcttagcttga

SEQ ID NO: 40 - protein, 341 aa, Pseudomonas stutzeri
MGRASLQPVIAVALAAALTGCLFQDKVEDFGGPTMGSTYSVKYVASDGAAAKAQLQRETESILAQLDEQLSTYRADSDIEAFNALPAGQCMAMPESARELVLAGQQLSQESDGALDLTIQPLLNLWGFGPQGRREQVPSAEEIARARETVGHRHLQVVGEQLCKDAAVEVDFNSIAAGYAVDLVAQRLEALGVESYLVEITGELKARGRKPGDAPWRIAIEAPRDNERVAQRVIELDGYGVSTSGDYRNYFERDGKRYSHTLDPQTGAPIEHRLAAVTVVDPSALRADGLSTVLMVLGPERGLAYAAEHRIAALFVIHEEQEFISKSTAAFDELFGAGAEQ

SEQ ID NO: 41 - DNA, 1026 bp, Pseudomonas stutzeri
  source 1..1026: mol_type = genomic DNA
atggggcgtgcgagcctgcaaccagtaattgctgttgctttagcggcggcgctgaccggttgccttttccaggataaggtcgaagattttggggggccgaccatgggtagtacttactctgtcaaatacgtcgccagcgatggcgcggctgcgaaagctcaactccaacgcgaaactgagagtatcttggcacagttggatgagcaactctccacttatcgtgctgacagcgatattgaagcctttaacgccctgccagctggtcaatgcatggcgatgccagaaagcgctcgcgaactggtattagcaggtcagcaacttagtcaagagtccgacggtgcgctggatctcacaatccaaccattactgaatttatggggtttcggccctcagggccgccgtgaacaagtcccatcggcagaggaaatcgctcgtgcccgtgagaccgtgggtcatcgtcatctccaggttgtgggcgaacaactgtgcaaggacgcagctgtcgaggtggattttaactcaattgcggcaggctatgctgtggacctcgttgcacagcgtcttgaagcgctcggtgttgagtcttatctggtggaaatcactggggaactgaaggcccgtggtcgcaaaccaggcgatgcgccgtggcgtatcgcgatcgaagctccgcgtgataacgagcgtgtggcgcagcgtgtaattgaattggatggttacggcgtcagcacctcaggtgactatcgcaactattttgaacgtgacgggaagcgttactctcacactctcgatcctcagaccggcgcacctatcgaacaccgcctggctgcggtaacggtggtagatccatcagctcttcgcgcagatgggttgtccactgtgcttatggtgctcggcccagaacgtgggttggcgtatgcggctgagcaccgtatcgcggcactctttgtgatccatgaggaacaggaatttatcagcaaatccactgccgcttttgacgagctcttcggggcgggggcagagcagtga

SEQ ID NO: 42 - DNA, 760 bp, synthetic construct
  source 1..760: mol_type = other DNA
ataacttcgtatagcatacattatacgaacggtacccatcagatccactagtggcctatgcggccgcggtgcgccgacatcataacggttctggcaaatattctgaaatgagctgttgacaattaatcatcggctcgtataatgtgtggaattgtgagcggataacaatttcacacaggaaacagaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagt

SEQ ID NO: 43 - DNA, 40 bp, synthetic construct
  source 1..40: mol_type = other DNA
agtggcctatgcggccgcggtgcgccgacatcataacggt

SEQ ID NO: 44 - DNA, 39 bp, synthetic construct
  source 1..39: mol_type = other DNA
tatttagaaaaataaacaaaaagagtttgtagaaacgca

SEQ ID NO: 45 - DNA, 30 bp, synthetic construct
  source 1..30: mol_type = other DNA
tttgtttatttttctaaatacattcaaata

SEQ ID NO: 46 - DNA, 30 bp, synthetic construct
  source 1..30: mol_type = other DNA
ccgcggccgcataggccactagtggatctg

SEQ ID NO: 47 - DNA, 39 bp, synthetic construct
  source 1..39: mol_type = other DNA
ttcacacaggaaacagaccatgcgcgcttgctacactct

SEQ ID NO: 48 - DNA, 39 bp, synthetic construct
  source 1..39: mol_type = other DNA
ccaaaacagccaagcttgctcaagctaagcgacgacgga

SEQ ID NO: 49 - DNA, 29 bp, synthetic construct
  source 1..29: mol_type = other DNA
gcaagcttggctgttttggcggatgagag

SEQ ID NO: 50 - DNA, 31 bp, synthetic construct
  source 1..31: mol_type = other DNA
gaattctgtttcctgtgtgaaattgttatcc

SEQ ID NO: 51 - DNA, 39 bp, synthetic construct
  source 1..39: mol_type = other DNA
tcacacaggaaacagaattcatggggcgtgcgagcctgc

SEQ ID NO: 52 - DNA, 38 bp, synthetic construct
  source 1..38: mol_type = other DNA
ccaaaacagccaagcttgcttatacgcgcccttctgcg

SEQ ID NO: 53 - DNA, 39 bp, synthetic construct
  source 1..39: mol_type = other DNA
tcacacaggaaacagaattcatgtccgacaatgacagca

SEQ ID NO: 54 - DNA, 39 bp, synthetic construct
  source 1..39: mol_type = other DNA
ccaaaacagccaagcttgcttaatggcctgcatggtcat

SEQ ID NO: 55 - DNA, 69 bp, synthetic construct
  source 1..69: mol_type = other DNA
tgtcacttcttctaatgaagtgaaccgcttagtaacaggacagattccgctaggtgacactatagaacg

SEQ ID NO: 56 - DNA, 69 bp, synthetic construct
  source 1..69: mol_type = other DNA
cttgcataaaaaagccaacccgcaggttggcttttctcgttcaggctggctcaagctaagcgacgacgg

SEQ ID NO: 57 - DNA, 20 bp, synthetic construct
  source 1..20: mol_type = other DNA
gctgtgccacgtttttatta

SEQ ID NO: 58 - DNA, 20 bp, synthetic construct
  source 1..20: mol_type = other DNA
ggtgaaagccgattgtgata

SEQ ID NO: 59 - DNA, 69 bp, synthetic construct
  source 1..69: mol_type = other DNA
gcgcgacccgttcgacgcgtaattctggtacgcctggcagatattttgcctaggtgacactatagaacg

SEQ ID NO: 60 - DNA, 69 bp, synthetic construct
  source 1..69: mol_type = other DNA
atcttgtgacatctcggttcctccgttgtcatctctgatgaagattttcgttatacgcgcccttctgcg

SEQ ID NO: 61 - DNA, 20 bp, synthetic construct
  source 1..20: mol_type = other DNA
tagagccgatttacgaaacc

SEQ ID NO: 62 - DNA, 20 bp, synthetic construct
  source 1..20: mol_type = other DNA
gtttggttaaatgttcgagg

SEQ ID NO: 63 - DNA, 69 bp, synthetic construct
  source 1..69: mol_type = other DNA
gacaccatcgaatggcgcaaaacctttcgcggtatggcatgatagcgccctaggtgacactatagaacg

SEQ ID NO: 64 - DNA, 69 bp, synthetic construct
  source 1..69: mol_type = other DNA
aagcctggggtgcctaatgagtgagctaactcacattaattgcgttgcgcttaatggcctgcatggtca

SEQ ID NO: 65 - DNA, 20 bp, synthetic construct
  source 1..20: mol_type = other DNA
gaatctggtgtatatggcga

SEQ ID NO: 66 - DNA, 20 bp, synthetic construct
  source 1..20: mol_type = other DNA
caaggcgattaagttgggta

SEQ ID NO: 67 - DNA, 70 bp, synthetic construct
  source 1..70: mol_type = other DNA
gatagcgcacaagctgcccgatatgcgtgaagtcgagccaggtttttaataggtgacactatagaacgcg

SEQ ID NO: 68 - DNA, 38 bp, synthetic construct
  source 1..38: mol_type = other DNA
ggaaacctgtcgtgccagatctcactatagggagaccg

SEQ ID NO: 69 - DNA, 400 bp, synthetic construct
  source 1..400: mol_type = other DNA
tctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattcgagctcggtacccggggatcctctagagtcgacctgcaggcatgca

SEQ ID NO: 70 - DNA, 38 bp, synthetic construct
  source 1..38: mol_type = other DNA
cggtctccctatagtgagatctggcacgacaggtttcc

SEQ ID NO: 71 - DNA, 70 bp, synthetic construct
  source 1..70: mol_type = other DNA
ttcgttcatcccgctcacaaagtaactctctggcttcaagcatacccacggtctgtttcctgtgtgaaat

SEQ ID NO: 72 - DNA, 20 bp, synthetic construct
  source 1..20: mol_type = other DNA
catgaaggacaataactcgc

SEQ ID NO: 73 - DNA, 20 bp, synthetic construct
  source 1..20: mol_type = other DNA
taacaggttttgatggtagc

SEQ ID NO: 74 - DNA, 39 bp, synthetic construct
  source 1..39: mol_type = other DNA
ttcacacaggaaacagaccatgaacttaattaagaagat

SEQ ID NO: 75 - DNA, 39 bp, synthetic construct
  source 1..39: mol_type = other DNA
ctcatccgccaaaacagccttataagccactcatatgct

SEQ ID NO: 76 - DNA, 39 bp, synthetic construct
  source 1..39: mol_type = other DNA
ttcacacaggaaacagaccatggggctggctcttgcatc

SEQ ID NO: 77 - DNA, 39 bp, synthetic construct
  source 1..39: mol_type = other DNA
ctcatccgccaaaacagccttatggcttcggataacctt

SEQ ID NO: 78 - DNA, 38 bp, synthetic construct
  source 1..38: mol_type = other DNA
ttcacacaggaaacagaccatgaaaaaaattctggtgc

SEQ ID NO: 79 - DNA, 38 bp, synthetic construct
  source 1..38: mol_type = other DNA
ctcatccgccaaaacagccttatttctgactcagcacc

SEQ ID NO: 80 - DNA, 39 bp, synthetic construct
  source 1..39: mol_type = other DNA
ttcacacaggaaacagaccatgaataacaaaaatccgat

SEQ ID NO: 81 - DNA, 39 bp, synthetic construct
  source 1..39: mol_type = other DNA
ctcatccgccaaaacagccttactgctttgcagcctcgt

SEQ ID NO: 82 - DNA, 38 bp, synthetic construct
  source 1..38: mol_type = other DNA
ttcacacaggaaacagaccatgaacaaaaacaacgtgc

SEQ ID NO: 83 - DNA, 38 bp, synthetic construct
  source 1..38: mol_type = other DNA
ctcatccgccaaaacagccttattcatccacatgcacg

SEQ ID NO: 84 - DNA, 39 bp, synthetic construct
  source 1..39: mol_type = other DNA
ttcacacaggaaacagaccatgaaattgaaacatttagc

SEQ ID NO: 85 - DNA, 38 bp, synthetic construct
  source 1..38: mol_type = other DNA
ctcatccgccaaaacagccttatttgccgccatccgcc

SEQ ID NO: 86 - DNA, 39 bp, synthetic construct
  source 1..39: mol_type = other DNA
ttcacacaggaaacagaccatggaaaaaaccatgctgaa

SEQ ID NO: 87 - DNA, 39 bp, synthetic construct
  source 1..39: mol_type = other DNA
ctcatccgccaaaacagccttagttgcccagcgccgcca

SEQ ID NO: 88 - DNA, 39 bp, synthetic construct
  source 1..39: mol_type = other DNA
ttcacacaggaaacagaccatgcgcattattgcgaaagg

SEQ ID NO: 89 - DNA, 39 bp, synthetic construct
  source 1..39: mol_type = other DNA
ctcatccgccaaaacagccttactgcagcgccaccgcca

SEQ ID NO: 90 - DNA, 38 bp, synthetic construct
  source 1..38: mol_type = other DNA
ttcacacaggaaacagaccatgtttcacaacagcctgg

SEQ ID NO: 91 - DNA, 39 bp, synthetic construct
  source 1..39: mol_type = other DNA
ctcatccgccaaaacagccttagttcacttgcgccagtt

SEQ ID NO: 92 - DNA, 39 bp, synthetic construct
  source 1..39: mol_type = other DNA
ttcacacaggaaacagaccatgattagcgcgaaaaccct

SEQ ID NO: 93 - DNA, 39 bp, synthetic construct
  source 1..39: mol_type = other DNA
ctcatccgccaaaacagccttattccacaatcactttgc

SEQ ID NO: 94 - DNA, 38 bp, synthetic construct
  source 1..38: mol_type = other DNA
taaggctgttttggcggatgagagaagattttcagcct

SEQ ID NO: 95 - DNA, 38 bp, synthetic construct
  source 1..38: mol_type = other DNA
catggtctgtttcctgtgtgaaattgttatccgctcac